(19) United States
(12) Patent Application Publication
(10) Pub. No.: US 2020/0105402 A1
(43) Pub. Date: Apr. 2, 2020

(54) NOTIFYING HEALTHCARE PROVIDERS OF FINANCIALLY DELINQUENT PATIENTS AND CONTROLLING HEALTHCARE CLAIMS

(71) Applicant: CIRRAGROUP, LLC, Lafayette, LA (US)

(72) Inventors: Wendell J. JUNEAU, Lafayette, LA (US); Mark G. FONTENOT, Lafayette, LA (US); Jared S. TESSIER, Lafayette, LA (US); Wendt L. WITHERS, Lafayette, LA (US); Lawrence C. BILLEAUD, Lafayette, LA (US); Michael V. DUCOTE, Lafayette, LA (US)

(21) Appl. No.: 16/288,414

(22) Filed: Feb. 28, 2019

Related U.S. Application Data
(63) Continuation of application No. 15/084,722, filed on Mar. 30, 2016, now abandoned.

(51) Int. Cl. G16H 40/20; G16H 10/60

(52) U.S. Cl. CPC G16H 40/20; G16H 10/60

(57) ABSTRACT

A database application of a platform receives patient data from providers and generates a database. The patient data includes identification and healthcare data of patients. A first application receives from providers first electronic queries of the database and generates from the patient data a first set of data comprising data of delinquent transactions. A second application receives from patients second electronic queries of the database and provides a second set of data and presents a payment interface configured to receive payments according to payment options. A third application receives from patients third electronic queries of the database and provides a third set of data along with finance options, and presents a finance interface configured to associate requesting patients with sources of debt financing for payment of the delinquent transactions.

### RELATED APPLICATIONS

This application claims the benefit of U.S. Patent Application No. 62/140,273, filed Mar. 30, 2015.

This application claims the benefit of U.S. Patent Application No. 62/145,680, filed Apr. 10, 2015.

This application is a continuation in part of U.S. patent application Ser. No. 14/171,479, filed Feb. 3, 2014, which is a continuation of U.S. patent application Ser. No. 12/886,058, filed Sep. 20, 2010, now U.S. Pat. No. 8,645,159.

This application is a continuation in part of U.S. patent application Ser. No. 14/213,845, filed Mar. 14, 2014.

This application is a continuation in part of U.S. patent application Ser. No. 14/215,332, filed Mar. 17, 2014.

### TECHNICAL FIELD

The present invention relates to electronically notifying healthcare providers of patients who have in the past been financially delinquent in their dealings with one or more other healthcare providers.

### BACKGROUND

Healthcare providers have learned that certain patients have a propensity to not pay for healthcare related services. These patients often see a number of different healthcare providers, failing to satisfy financial obligations to one or more of them. Conventional systems used by healthcare providers include systems and methods for credit evaluation, healthcare related services and payment for said services, payment management, and collection of delinquent debts that are related to healthcare service providers. However, conventional systems do not address notifying healthcare providers of patients who have been financially delinquent in one or more patient care related transactions with another healthcare provider.

### INCORPORATION BY REFERENCE

Each patent, patent application, and/or publication mentioned in this specification is herein incorporated by reference in its entirety to the same extent as if each individual patent, patent application, and/or publication was specifically and individually indicated to be incorporated by reference.

### BRIEF DESCRIPTION OF THE DRAWINGS

FIG. 1 depicts a traditional healthcare timeline and flow of accounts.

FIG. 2 shows a healthcare revenue cycle environment including the patient-debtor service (PDS), under an embodiment.

FIG. 3A is a block diagram of an example patient-debtor system (PDS) for tracking information of and notifying healthcare providers of financially delinquent patients, under an embodiment.

FIGS. 3B-3E are example subscriber or user interface pages presented by the platform to each subscriber by which the subscriber accesses the platform and database, under an embodiment.

FIG. 4 is a flow diagram for tracking and notifying healthcare providers of financially delinquent patients, under an embodiment.

FIG. 5 is a flow diagram for facilitating and collecting payment from a patient debtor on behalf of a subscriber, under an embodiment.

FIG. 6 is an example of patient information of the system database, under an embodiment.

FIG. 7 is a block diagram of a healthcare revenue environment in which the patient-debtor service (PDS) is provided to healthcare providers through an intermediate billing company, under an embodiment.

FIG. 8 is a block diagram of a healthcare revenue environment in which the patient-debtor service (PDS) is provided directly to each healthcare provider, under an alternative embodiment.

FIG. 9 is a block diagram showing classes of subscribers to the patient-debtor service (PDS), under an embodiment.

FIG. 10 describes the debt resolution process of the PDS after a subscriber notifies the patient of their delinquent status, under an embodiment.

FIG. 11 is a flow diagram for distributing revenue collected via the PDS, under an embodiment.

FIGS. 12A-B are a flow diagram for collecting debts relating to healthcare services via the PDS platform and/or a collection agency, under an embodiment.

FIG. 13 is a flow diagram for collecting debts relating to healthcare services using the PDS platform as a data warehouse, under an embodiment.

FIG. 14 is a block diagram of a patient-debtor system (PDS) that includes the patient debt tracking system (DTS) and a collection management system (CMS), under an alternative embodiment.

FIG. 15 is an example user interface including threshold-setting controls for collection management parameters, under an embodiment.

FIG. 16 is an example user interface including debt depreciation controls for collection management parameters, under an embodiment.

FIGS. 17A-B are another example user interface including debt depreciation controls for collection management parameters, under an embodiment.

FIG. 18 is a flow diagram between a healthcare provider and the payer for the 270/271 transaction.

FIG. 19 is a flow diagram of the 270/271 process between the healthcare provider and the payer via a clearinghouse.

FIG. 20 is a block diagram of the PDS coupled with a 270/271 HETS process in which the PDS and the payer transact with the 270/271, under an embodiment.

FIG. 21 is a block diagram of the PDS coupled with a 270/271 HETS process in which the PDS is an intermediary in the 270/271 process, under an embodiment.

FIG. 22 is a block diagram of the PDS coupled with a 270/271 HETS process in which the PDS is integrated into a clearinghouse that intermediates the combined PDS inquiry and 270 request, under an embodiment.

FIG. 23 is a block diagram of the PDS coupled with a 270/271 HETS process in which the PDS and a clearinghouse separately intermediate the combined PDS inquiry and 270 request, under an embodiment.

FIG. 24 shows data of an example scenario for the PPCM score and expected dollar payment from a patient, under an embodiment.

FIG. 25A is a flow diagram of the Direct Insurance Recovery Cycle (DIRC) enabled by the system or platform, under an embodiment.

FIG. 25B is a block diagram of the system or platform for the DIRC, under an embodiment.

FIG. 26 is an example schedule of DIRC account reconciliation for automated or selected factoring, under an embodiment.

FIGS. 27A-B are an example schedule accounting transactions of DIRC account reconciliation for automated or selected factoring, under an embodiment

FIG. 28 is a block diagram of the Direct Consumer Debt Revenue Cycle (DCDRC) enabled by the platform, under an embodiment.

FIG. 29 is a block diagram of the Patient Financing System (PFS), under an embodiment.

FIG. 30 is an example electronic CAF of the PFS, under an embodiment.

FIG. 31 is an example PFS presentation of the results of the PFS process, under an embodiment.

FIG. 32 is a flow diagram for Selective Processing of the PFS, under an embodiment.

FIG. 33 is a flow diagram of FICO Score Filtering of the PFS, under an embodiment.

FIG. 34 is a flow diagram of unbundling and selective processing of healthcare treatment plans using the PFS, under an embodiment.

FIG. 35 is a block diagram of a provider service example using the provider revenue cycle combining DIRC and DCDRC enabled by the platform, under an embodiment.

FIG. 36 shows an example of a patient seeking the services of a provider that is recommending a $5,000 treatment plan, $1,000 of which is covered by the patient's medical insurance plan and the remaining $4,000 would be required to be covered by the patient as an out-of-pocket cost, under an embodiment.

FIG. 37 is a block diagram of patient revenue cycle platform, under an alternative embodiment.

### DETAILED DESCRIPTION

A database application of a platform receives patient data from providers and generates a database. The patient data includes identification and healthcare data of patients. A first application receives from providers first electronic queries of the database and generates from the patient data a first set of data comprising data of delinquent transactions. A second application receives from patients second electronic queries of the database and provides a second set of data and presents a payment interface configured to receive payments according to payment options. A third application receives from patients third electronic queries of the database and provides a third set of data along with finance options, and presents a finance interface configured to associate requesting patients with sources of debt financing for payment of the delinquent transactions.

The American Hospital Association (AHA) has reported that uncompensated care cost hospitals more than $326 billion from 2000 to 2010. Uncompensated service can be divided into two categories: bad debt and charitable care. A balance that could have been collected, but was not, is considered a bad debt. Hospitals administer charitable care without the expectation of payment when a patient fails to meet the criteria for being able to pay. Escalating personal healthcare costs and a growing self-payer population fuel the upsurge in bad debt and charitable care.

In 2010 alone, uncompensated care was $39.3 billion and accounted for 5.8% of hospital expenses. A majority of administered care expenses at the average hospital are reimbursed by Medicare or Medicaid and Federal law requires annual adjustment of Medicare reimbursement rates to maintain solvency. The decrease in Medicare reimbursements coupled with the rise in uncompensated care directly affects the profitability of medical institutions. Approximately 30% of all US hospitals operate with negative profit margins. Moreover, uncompensated care is not a problem unique to hospitals as it permeates the entire healthcare system. It is projected that half of all physicians in the US operate a private practice, and it is not uncommon for an independent private practice to suffer about 10% to 15% or more profit loss on average from unpaid debt. Thus, these small practices are extremely sensitive to this type of profit leakage.

Additionally, the cost of insurance premiums and out-of-pocket medical expenses has outpaced inflation and wages. The self-payer community includes uninsured and underinsured patients. In 2009, the roles of the non-elderly uninsured in the US grew to an estimated 49.1 Million. nTelagent, a company that provides integrated point-of-service solutions for managing accounts receivable (A/R), contributed 65% of bad debt to insured patients in 2008. A fifth of America's workforce has high-deductible employee-sponsored insurance coverage. In these plans an individual pays a deductible greater than $3,000 and more than $6,000 for a family.

As the healthcare industry matures, the diverse set of business processes and services undergo consolidation. Health IT platforms like patient portals and electronic health records (EHRs) have emerged to boost efficiency and increase data sharing. The HITECH Act, part of the American Recovery and Reinvestment Act, made incentives available to doctors and hospitals to adopt health IT and EHRs. As of 2011, health IT has been adopted by 35 percent of US hospitals with 85 percent of hospitals reporting that they intend to take advantage of EHR incentive programs by 2015. To date, a total of $3.1 billion has been distributed to more than 43,000 providers migrating to this technology. EHRs simplify documentation, reduce mistakes, and give any provider access to a patient's complete medical history, thereby enabling healthcare providers to deliver superior care using less time and money. EHRs also give patients the ability to verify the accuracy of their medical records a very important part of tracking and repairing medical identity theft.

Healthcare providers have learned that certain patients have a propensity to not pay for healthcare related services. These patients often see a number of different healthcare providers, failing to satisfy financial obligations to one or more of them. A provider's good-faith, credit extension is routinely abused by patients that have the capacity to repay but for whatever reason choose not to. Healthcare providers that attempt to stop this type of abuse often find themselves hamstrung by laws enacted to protect the consumer. If caution is not exercised during the adjudication process, they may face serious consequences if found in violation.

The most straightforward solution to curb abuse involves requesting a patient's credit score before service and reporting any delinquent debt to credit bureaus afterwards. A credit score in the United States is a number representing the creditworthiness of a person or the likelihood that person would pay his or her debts. The credit score has been demonstrated to be very predictive of risk and, therefore, has made credit more widely available to consumers and lowered the cost of providing credit. A credit score is primarily based on a statistical analysis of a person's credit report information, typically from the three major American credit bureaus, namely Equifax, Experian, and TransUnion. Lenders, such as banks and credit card companies, use credit scores to evaluate the potential risk posed by lending money to consumers and to mitigate losses due to bad debt. Because a score does not consider race, sex or ethnicity, it is generally considered to be the most fair and objective underwriting tool available to lenders. A study conducted by the Federal Reserve Board noted that credit scores have increased the availability of credit and reduced the cost of credit. Scores have also proven to be very predictive in assessing risk.

In practice, the use of conventional credit reporting to pre-screen patients increases the burden of regulatory compliance for the provider. There is also a movement to stop medical debt from impacting an individual's financial health. For example, checking a patient's credit report before rendering service can require Address Discrepancy and Red Flag Rule compliance as set forth in the Fair Credit Report Act (FCRA). Even the simple act of pulling a patient's credit report can lower their credit score. At the opposite end of the revenue cycle, if a provider chooses to report all bad debts to credit bureaus, patients can suffer far-reaching financial consequences. Taking any action that might damage a patient's credit score is becoming a less attractive option given growing public sentiment that deems medical debt involuntary. This viewpoint is apparent in the Medical Debt Responsibility Act that, if enacted by Congress, would require credit bureaus to remove paid debt less than $2,500 within 45 days. Preempted by legal liability and adverse public relations, healthcare providers are left with little recourse to halt abuse or recover debt.

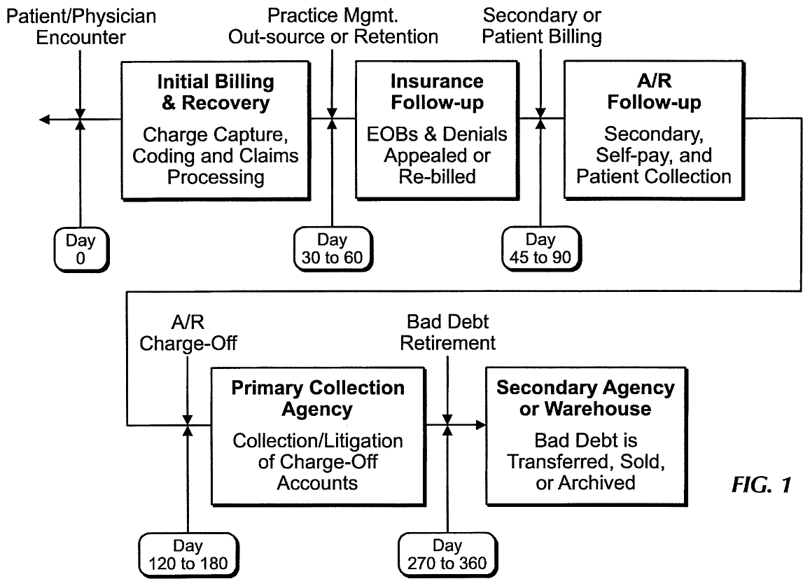

Accounts Receivable (A/R) days measure the average time a balance remains an accounts receivable until collection. FIG. 1 depicts a traditional healthcare timeline and flow of accounts. Providers try to decrease A/R days at Day 0 by appropriately collecting copay amounts, discounting self-payers for advanced payment in full, and checking a patient's insurance eligibility. During the initial billing and recovery, the daily filing of insurance claims and transmission of statements can further decrease A/R days. During the period of day 30 to 60, accounts move to insurance follow-up in which efficiency is highly dependent on quick denial turn around and consistent follow-up. If the provider employs business process outsourcing (BPO), accounts are outsourced at this time to fulfill practice management and other back office functions. During the period of day 45 to 90, secondary payers and self-payers are billed. During the period of day 120 to 180, accounts are charged-off and sent to the primary collection agency. Once the debt age reaches 270 to 365 days, it is retired to a data warehouse or referred to a secondary collections agency. Some hospitals have used the sale of A/R as a way to increase liquidity and optimize their revenue cycle. In this case, a third-party values the debt at the charge-off or retirement day and brokers a deal to collect the bad debt instead of the hospital.

An effort to prevent bad debt at the front-end has led to the advent of specialized pre-screening services. One such service is nTelagent described above, which verifies insurance and ascertains the patient's ability to pay before services are rendered. Publicly available information is searched and aggregated in order to classify the patient as belonging to a particular demographic. Information used to determine ability to pay could be annual household income or the make/model of the guarantor's vehicle. Patients are identified by nTelagent using their name and address. By assessing risk based solely on publicly held data, the FCRA does not apply, so this system can prove valuable in assessing individuals with no credit history. However, there may be limited information known about individuals that move frequently.

The intense investigation and scrutiny used to classify patients in such a product could raise concerns of privacy, socio-economic bias and geographical bias. A credit reporting process that forecasts an individual's future financial performance based solely on their payment history is fairly objective, whereas, demographic information can be used in very subjective ways. Might a patient be treated differently because they live in a low-income area of town, lack a degree in higher education, or have had a failed marriage?

Another objective of self-pay management systems (e.g., nTelagent) is that they shift self-pay collection to the point of service (POS) before a patient receives medical services. These systems estimate the cost of medical service including any amount reimbursed by payers and collect payment in full, or provide financing in advance using a provider of consumer debt, for example the collection management system of the patient-debtor service (PDS) of an embodiment described herein. Either way the provider is compensated at the very beginning of the revenue cycle, thereby eliminating risk. However, the fact still remains that even with financial assistance some patients simply cannot cover all charges upfront. Additionally, accurate estimation of patient liability in all cases is not feasible. In these circumstances, the status quo of backend collection serves the consumer quite well. Many health providers want to extend credit to patients in need of medical treatment. However, they do not want this policy to be abused by individuals that cannot be tracked.

In summary, rising healthcare and insurance costs continue to increase the number of so-called self-payers. As a consequence, bad debt management becomes paramount as profit margins shrink for hospitals and independent practices, alike. The ability to measure, benchmark, and lower A/R days can dramatically increase revenue. However, the industry lacks real-time benchmarking tools that do not rely on surveys and questionnaires. Progressive patient's rights laws in some states can severely lower collections ratios of conventional debt collection agencies. As healthcare intuitions consider selling debt to third parties, the accurate valuation of debt becomes a challenge.

The use of credit evaluation in the financial sector has proven very successful in determining credit risk and lowering the cost of credit. Using the same tool in the medical industry to assess credit risk during patient access does not yield the same benefits. In fact, it only increases the burden of regulatory compliance. The complex healthcare revenue cycle and sensitive nature of medical data has created a fragmented environment with restricted sharing of information. This closed-off system makes abuse of backend collections and medical identity theft impossible to track.

Conventional systems used by healthcare providers include systems and methods for credit evaluation, healthcare related services and payment for the services, payment management, and collection of delinquent debts that are related to healthcare service providers. However, conventional systems do not address notifying healthcare providers of patients who have been financially delinquent in one or more patient care related transactions with another healthcare provider.

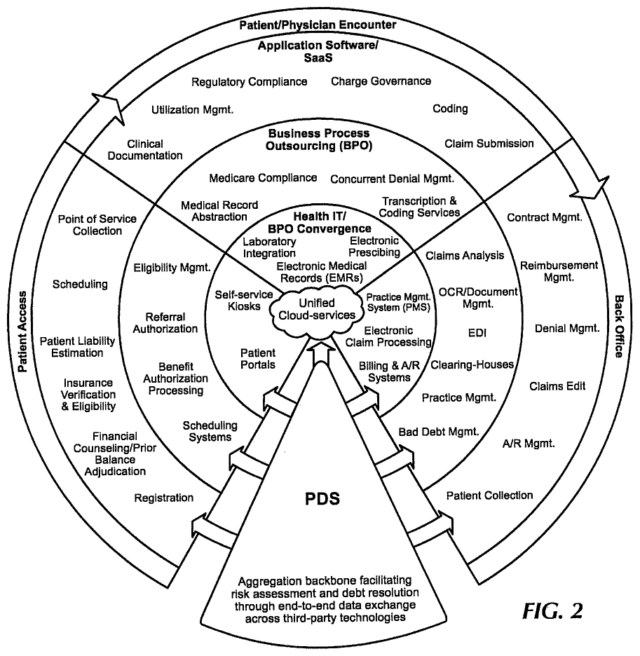

Embodiments provide for tracking and notifying healthcare providers of financially delinquent patients using a platform that enables risk assessment and debt resolution through end-to-end data exchange across third-party technologies. FIG. 2 shows a healthcare revenue cycle environment including the patient-debtor service (PDS), under an embodiment. The PDS includes a PDS platform or service platform (also referred to herein as “the platform”) coupled to a database including identifying data of patient debtors who have been financially delinquent in a healthcare transaction with a healthcare provider. The identifying data (also referred to herein as patient debtor data) includes patient and financial identification data of the transaction.

The identifying data described herein includes Personally Identifiable Information (PII) and Protected Health Information (PHI). The PII includes any information that can be used to identify, contact, or locate an individual, either alone or combined with other easily accessible sources, and includes information that is linked or linkable to an individual, such as medical, educational, financial and employment information (e.g., name, fingerprints or other biometric (including genetic) data, email address, telephone number, social security number, etc.). The PHI includes any information about health status, provision of health care, or payment for health care that can be linked to a specific individual, and includes any part of a patient's medical record or payment history. PHI is a subset of PII in that a medical record can be used to identify a person.

Subscriber information is received from a subscriber (e.g., healthcare provider, patient, etc.) that enables the subscriber to electronically access the platform. The subscriber information includes identifying information of the subscriber. Additionally, when the subscriber is a healthcare provider, the subscriber information may include data of patient debtors of that subscriber. A subscriber also may pay a fee to access the platform under alternative embodiments. The identifying data of patient debtors is provided to the subscriber via the platform. New identifying data of patient debtors is received at the platform from a contributing subscriber (healthcare provider). The new identifying data is identifying data of a new patient debtor and/or a new healthcare transaction of an existing patient debtor.

Embodiments described herein provide systems and methods for notifying healthcare providers of patients who have been financially delinquent in one or more patient care transactions with healthcare providers. Generally, a service platform including or coupled to a database is accessible via an Internet website hosted by a service company or provider. The database includes data of patient debtors who have previously been financially delinquent in one or more patient care related transactions with one or more healthcare providers. Subscribing healthcare providers, also referred to herein as subscribers or members, may pay a fee for accessing the information via the website and service platform. Each contributing subscriber that contributes data to the database of financially delinquent patients may receive a credit toward the membership fees or other fees that are collected by the platform.

To manage bad debt incurred by the growing uninsured and underinsured self-payer population, healthcare providers assess financial risk and present up-front costs before administering care. When using conventional techniques to assess risk a healthcare provider must use the limited scope of their own aged receivables, the individual's FICO score, or classify the patient using demographic information. Embodiments described herein enable patient pre-screening using a history of only outstanding medical debt and aids in resolving medical bad debt, specifically. Instead of using demographic information that may not accurately gauge a patient's financial risk, the system generates a score based on delinquent debt and payment history. However, because medical debt can be involuntarily, the embodiments serve to insulate a patient from the punitive consequences of reporting the debt to credit bureaus. The embodiments herein therefore encourage medical debt resolution without the need for bankruptcy.

Embodiments enable only authorized subscribers to access the database via the user interface and platform. Each subscriber may pay a membership fee to the service company for the privilege of accessing the database. When a membership fee is charged, the fee can be a periodic subscription fee (e.g., monthly fee, yearly fee, etc.), a transaction fee charged per transaction (each time the subscriber accesses the database the individual request is billed on a per click/use basis), or a combination of a subscription fee and a transaction fee. Regardless of the type of membership fee charged, the membership fee can be a fixed fee, a variable fee, or a combination of fixed and variable fees.

Embodiments of the systems and methods encourage subscribers to contribute new information to the PDS database (service company), where the new information identifies additional patient debtors that are financially risky. This new information can include the name and address of one or more new financially risky or delinquent patients in addition to the amount of any outstanding unpaid balance for healthcare services administered. A subscriber that reports new information to the service company that identifies a particular new patient debtor as a financial risk could in an embodiment be rewarded with a discount that lowers the membership fee (e.g., subscription fee and/or transaction fee) charged that subscriber.

Embodiments include a web-based data resource or management platform that provides healthcare providers a mechanism to limit treatment of non-paying patients. An embodiment is supported by a contract between each healthcare provider and the healthcare credit service company or other third party hosting the service platform. The subscriber may pay a membership fee for the service (e.g., yearly fee, with the first year free, etc.). When received, payments from a subscriber serve to open an account, create an account, and maintain an account for that subscriber. The payments of an embodiment are in the form of electronic transmissions, automatic debit or credit card transactions, and/or direct withdrawal from a subscriber-identified account. A statement of debits/withdrawals from a subscriber account is electronically provided to each subscriber on a periodic basis (e.g., weekly, monthly, quarterly, etc.). The website is secure and available for limited viewing by subscribers only, and includes mechanisms to protect the security/identity of both subscribing healthcare providers and debtor-patients.

Embodiments of the PDS described herein effect the provision of notice to healthcare providers regarding whether potential and/or new patients are a financial risk by having subscribers input or upload identification information of delinquent patients on a periodic basis (e.g., daily, weekly, monthly, etc.) to the service platform for each new patient it deems to be a financial risk to other healthcare providers. The information uploaded for a delinquent patient can include, for example, social security number and the amount of indebtedness, but is not so limited. The healthcare provider could be given a partial financial credit towards its membership fees in exchange for information provided relative to new non-paying patients.

Each healthcare provider has a secure and unique access code for the website, with tracking mechanisms within the database to identify the source of the information received. Similarly, the patients are identified with a patient identification number (e.g., social security number, assigned identification number, etc.). In an embodiment, each subscriber receives a monthly report that includes a summary of information related to delinquent patient debtors. Negative financial reports are removed from the database within a specified period of time (e.g., 24 hours) following receipt of notice of final payment on account from a subscribing creditor/healthcare provider.

Each subscriber, following registration, is allowed to access the database to verify which patient debtors have been financially delinquent or are financially risky. The subscriber of an embodiment is not charged a subscription fee, but instead may provide other consideration. In an alternative embodiment, a subscriber pays a periodic fee (e.g., monthly, yearly, etc.) or fee based upon a flat rate. In another embodiment, a subscriber pays a fee each time that subscriber accesses the database. In yet another embodiment, a subscriber pays both a periodic fee and an access fee each time that subscriber accesses the database. Regardless of the fee structure under which a subscriber is billed, the subscriber could also obtain a discount that lowers the fees in exchange for the reporting of information evidencing that a particular patient debtor presents a financial risk for other subscribers. The information contributed can include the amount of an unpaid indebtedness. The database is updated periodically (e.g., daily, weekly, etc.) to remove information of any patient debtor who has satisfied his or her indebtedness to a subscriber for a past healthcare related transaction. For at least some patients included in the database, the database includes a monthly summary of debits and withdrawals from an account.

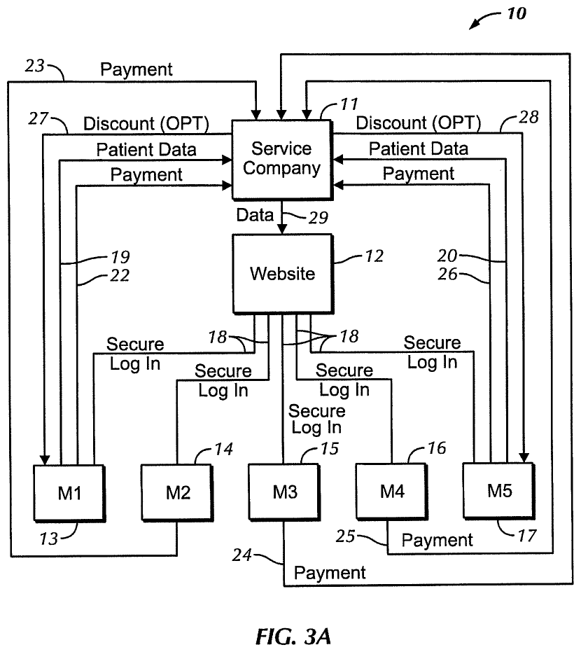

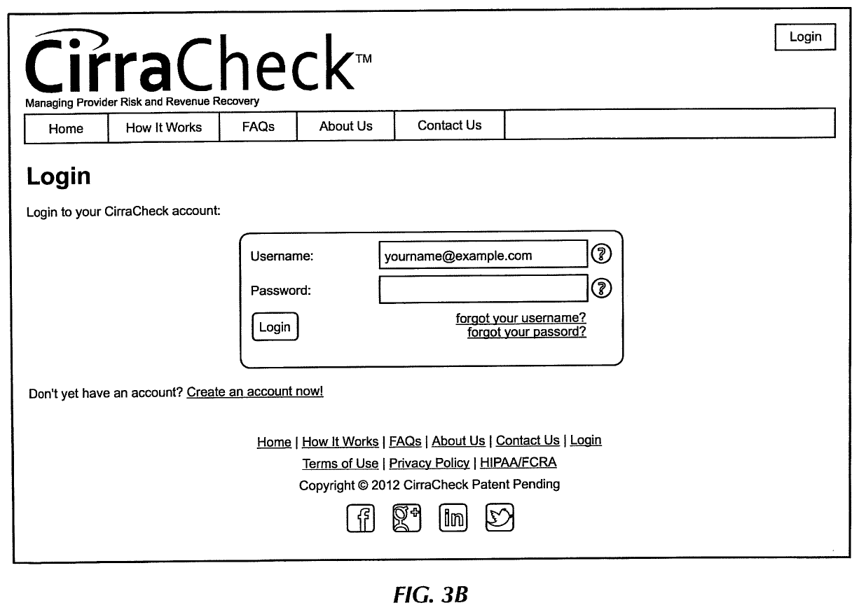

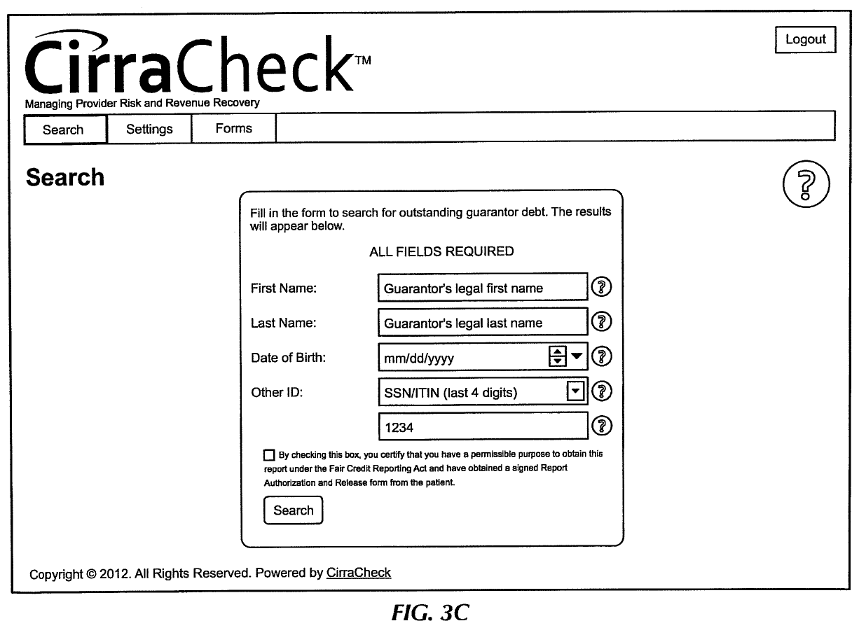

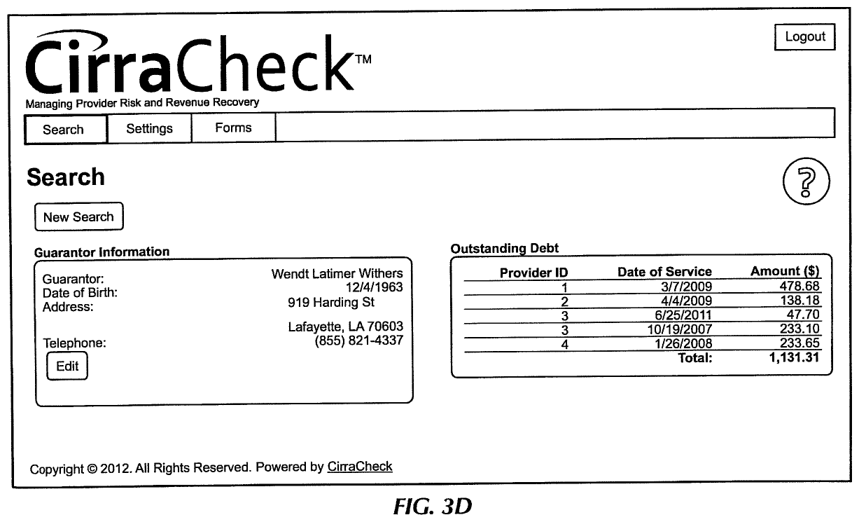

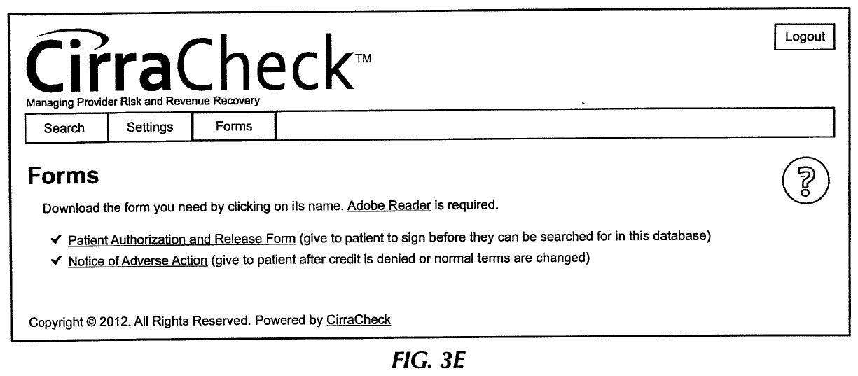

FIG. 3A is a block diagram of an example patient-debtor system (PDS) 10 for tracking information of and notifying healthcare providers of financially delinquent patients, under an embodiment. The PDS 10 includes a service platform 11 that is coupled to or includes a website 12 (e.g., network-based website, Internet-based website, etc.). The service platform 11 is hosted by a service provider that is a third party company, but is not so limited. A number of subscribers are coupled to the platform 11 via the website 12 such that the subscribers can access information of patient debtors. The PDS, which includes at least the debt tracking system and can include other systems or components as described herein, includes and/or is coupled to a database that includes identifying data of patient-debtors who have been financially delinquent in a healthcare transaction with a healthcare provider.

The database comprises patient information, also referred to as patient debtor information that includes but is not limited to one or more of the full names, addresses, social security numbers, date of birth, a unique identification number of the patient debtor (e.g., driver license number, state identification card number, passport number, etc.), name and address of healthcare provider including national provider identifier, patient account history, and past account data for patients who have been identified as financially delinquent. For example, the patient information can include information of patient Jane Doe that includes one or more of her full name, address, contact information (e.g., telephone number(s), email address(es), etc.), social security number, date of birth, name and address of healthcare provider including national provider identifier, date(s) healthcare services were rendered, patient account history, past account data, and any outstanding amounts not paid to the healthcare provider. As another example, the patient information can include information of patient John Smith that includes one or more of his address, telephone number, previous healthcare providers who have rendered healthcare services to John Smith, dates healthcare services were rendered, and any outstanding amounts not paid to the healthcare providers.

In the example system 10, five subscribers or users (M1-M5) 13, 14, 15, 16, 17 are coupled or linked 18 to the platform 11 via the website 12 and a client device, but the embodiment is not limited to five subscribers. The link 18 enables each subscriber 13-17 to access the platform 11 and its database via a user interface presented to the user via the website 12 and a client device, and the transfer of patient information between the subscribers 13-17 and the database occurs over the link 18. Access to the database is accomplished with a coded login such that each subscriber 13-17 is given a different, confidential password or code. When the subscriber 13-17 accesses the platform 11 via website 12, that subscriber (such as subscriber 13) is able to obtain information included in the database via the website 12. A data transfer 29 occurs between the platform 11 and the website, and this supports provision of information to the subscribers 13-17 via a subscriber interface. FIGS. 3B-3E are example subscriber or user interface pages presented by the platform to each subscriber by which the subscriber accesses the platform and database, under an embodiment.

Each of the subscribers 13-17 of this example pays a fee 22-26 (e.g., yearly, monthly, per-access, other agreed fee, etc.) to the service company as a prerequisite to accessing the platform 11, but the embodiment is not so limited. This fee payment 22-26 can be a fixed or variable payment per time period. Additionally or alternatively, the payment 22-26 can also be a payment that is due each time that a subscriber or user 13-17 accesses the database of the website 12.

Subscribers 13-17 of an embodiment provide updated data to the service platform via the website 12, and the updated data is added to the information of the database. In providing the data to the PDS, the provider can provide their data directly to the PDS; alternatively, the provider's data can be provided to the PDS via a third party, for example a billing company that performs billing services on behalf of the provider. When a subscriber 13-17 does supply such updated information, and the subscriber is begin charged a subscription fee, that subscriber 13-17 may receive a discount of the subscription fee. In the example system 10, there is an example transfer of data 19 from subscriber 13 to the service company 11. Another example is a transfer of patient data 20 from the subscriber 17 to the service platform 11. By providing a discount 27, 28, each subscriber 13-17 could be encouraged to help maintain a current and updated database for the website 12. This updated information of patient data 19, 20 insures updated information for all of the subscribers 13-17.

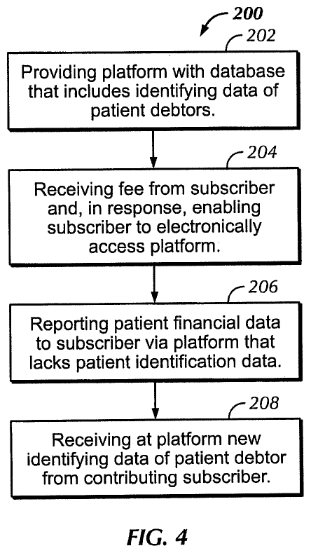

FIG. 4 is a flow diagram for tracking and notifying healthcare providers of financially delinquent patients 200, under an embodiment. Embodiments provide a platform with a database that includes identifying data of patient debtors 202. The patient debtors are patients who have been financially delinquent in at least one healthcare transaction with at least one of a number of healthcare providers. The identifying data includes patient identification data and financial data of at least one healthcare transaction. Embodiments receive subscription information and, optionally, a fee from a subscriber and, in response, enable the subscriber to electronically access the platform 204. The subscriber is a healthcare provider of the plurality of healthcare providers. Embodiments provide the identifying data of at least one patient debtor to the subscriber via the platform 206. Embodiments receive at the platform new identifying data of at least one patient debtor from a contributing subscriber of the healthcare providers 208. The new identifying data of at least one patient debtor and a new healthcare transaction of an existing patient debtor correspond to at least one healthcare transaction with the contributing subscriber. Embodiments could automatically apply a discount to the at least one fee of the contributing subscriber in response to receipt of the new identifying data 210.

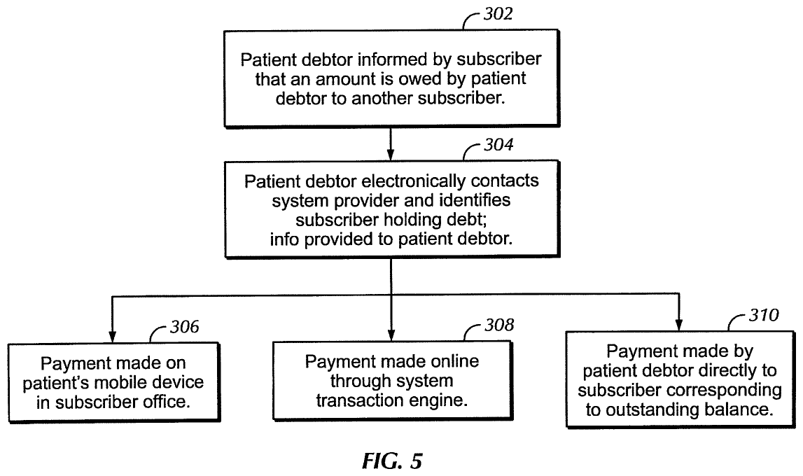

FIG. 5 is a flow diagram 300 for facilitating and collecting payment from a patient debtor on behalf of a subscriber, under an embodiment. Once a healthcare provider subscriber has identified a patient as owing a balance to another healthcare provider subscriber 302, the identified patient debtor can electronically contact (e.g., electronic link, network connection, electronic mail, telephone call, etc.) the service provider to identify the subscriber to which the patient debtor owes the outstanding balance 304. During the electronic contact or session, the patient debtor confirms their identity with their name and social security number (can include additional identifying information (e.g., date of birth, etc.)). The system database includes but is not limited to the subscriber's identification, the patient debtor's Social Security number, the date the debt was entered into the database, and the amount of the debt, for example. The service provider provides information to the patient debtor to contact the PDS to identify the other healthcare provider(s) who is(are) owed money.

The system of an embodiment provides the patient debtor with a number of payment options 306-310 if the patient debtor elects to pay some or all of the balance owed to the subscriber. Under one payment option, the patient debtor can make a payment at the subscriber's office in which the patient debtor is seeking treatment; under this option the patient debtor can use a credit card payment terminal provided by and coupled to the system 306. Under another payment option, the patient debtor can make a payment online at the subscriber's office in which the patient debtor is seeking treatment; under this option the patient debtor can use a system web portal with is coupled to and provides access to the transaction engine 308. Under yet another payment option, the patient debtor can contact the subscriber owning the outstanding balance and make arrangements for direct payment of the subscriber (e.g., check, cash, credit card, financing, etc.) 310. When the patient debtor makes a payment via credit card or online at the subscriber's office in which the patient debtor is seeking treatment, the system disburses the amount paid by the patient debtor to the subscriber owning the patient debtor balance, less a transaction fee.

While the PDS of an embodiment contemplates that a patient debtor wanting to settle a delinquent account contacts the corresponding healthcare provider regarding payment arrangement, the PDS of an alternative embodiment enables any subscriber to collect a debt on behalf of any other healthcare provider subscriber that is owed money. Regardless of where the payment is made, immediate payment of bad debt by a creditor has the affect of “clearing” this negative entry in the system database upon receipt of payment. This immediate removal of the negative entry provides the debtor the incentive to make payment, as he/she will receive an immediate benefit, unlike traditional credit databases. Thus, membership authorizes the system of an embodiment to act as a collection company. Payment to the subscribers may be split on a pro rata basis, or some other method that the patient may choose, should the debt not be cleared entirely. Pursuant to the subscriber agreement, the system is authorized to retain as a service fee, a pre-specified percentage of funds collected in this manner and/or a flat transaction fee.

In an alternative embodiment, the service provider grants the patient debtor access to the system website by assigning the patient debtor a website access key code. Using this key code, the patient debtor is granted access to the website. On the entry page of the website, a transaction button can be clicked that takes the patient to the transaction engine. The database information is displayed on the transaction page. The patient debtor can select a payment method. If the payment is made by credit card, the patient debtor enters their credit card number, expiration data, credit card security code, and amount of payment. The credit card information is processed and a page confirms the amount to be paid and any outstanding balance remaining after the payment is applied. After payment is made, the transaction engine disburses the payment to the subscriber to which the balance is owed less a transaction fee. In addition, the database is updated to reflect the payment.

Regarding the data of delinquent patient debtors provided under the embodiments herein, it is noted that a reporting company, such as the service provider described herein, that provides information to health service providers regarding an individual's health services liabilities likely falls within the scope of the Fair Credit Reporting Act (“FCRA”). As such, the FCRA restricts a reporting agency's ability to report health care related data, effectively limiting the content of such reports to the existence of outstanding account balances relating to the provision of medical services.

Consider the example case of a patient requesting the services of a physician. Prior to accepting the patient or establishing any relationship with the patient, the physician requests information from the service provider regarding the individual's outstanding health services accounts. The physician subsequently uses the information to determine the patient's ability to pay and, based on the information, the physician may decide to demand cash payment prior to treatment or provide treatment with the patient's commitment to pay at a later date. In either case, the physician has used the information to determine whether to issue the patient credit as the term in defined in the FCRA (e.g., § 603(r)(5)).

The FCRA governs the conditions under which the service provider may provide a patient's or consumer's health related account history to requesting health care providers. In more general terms, the FCRA governs a consumer reporting agency's ability to issue consumer reports (e.g., § 603(f)). Under the FCRA, a consumer reporting agency is broadly defined to include any person who collects credit information for the purpose of providing consumer reports. The term consumer report constitutes any communication generally related to an individual's credit worthiness and used as a factor in determining such individual's eligibility for credit (e.g., § 603(d)).

The service provider must comply with the FCRA in providing physicians or other healthcare providers consumer data. The FCRA generally authorizes a consumer reporting agency (i.e., the service provider) to provide consumer reports to a person that (in the reasonable belief of the issuing party) intends to use the information with respect to a credit transaction involving the consumer (e.g., § 604(a)(3)(A)) or an otherwise legitimate business need in a business transaction initiated by the consumer (e.g., § 604(a)(3)(F)(i)).

However, the FCRA aggressively protects consumer medical data and imposes strict requirements upon a consumer agency's use and dissemination of medical data. In particular, the FCRA specifically excludes certain medical information from consumer reports unless expressly authorized under the statute. Without consumer consent, the FCRA restricts the reporting of medical information to general transaction and account balance data using codes that do not disclose any service provider or the nature of the services themselves (e.g., § 604(g)). In addition, the FCRA prohibits the reporting of the name, address and telephone number of any medical information furnisher unless the information is reported using codes that do not provide sufficient information to infer the identity of the health care provider or nature of the services (e.g., § 605(a)(6)). The embodiments described herein, when in actual use, comply with the FCRA as to the disclosure of patient financial information.

The FCRA (e.g., § 603(i)(1) and § 603(i)(2)) does not consider identifying information, insurance policy, or other irrelevant data as “medical information.” However, medical payments are considered “medical information.” Under FCRA (e.g., § 603(x)), the provider of the PDS is defined as a “Nationwide Specialty CRA” because it reports medical charges. As such, (e.g., § 612(a)(1)(C)), the provider of the PDS is required to make free annual file disclosures through a streamlined process to consumers who submit requests directly to the PDS. The reporting of medical information such as consumer medical debts is allowed without consent (e.g., § 604(g)) if the information is coded in a manner that conceals the provider identity and nature of service.

The patient identification data of an embodiment comprises at least first name, middle name (if applicable), last name, date of birth, social security number, complete current address with zip code, and identification card number (e.g., driver license number, state identification card number, military identification card number, passport number, etc.). Personal identifiers are keyed in a masked search field to locate any existing patient records. To limit the exposure of sensitive information, these personal identifiers are not shown in search results or reports. The PDS uses a unique identification number to identify the source of the debt being reported by the PDS; this unique identification number is used to protect the true identity of the corresponding healthcare provider or the nature of the service provided. This information is available to the patient after verifying their identity by a representative of the call center or through the patient web application.

A disclosure pertaining to the use of the PDS is provided (e.g., electronic disclosure form) to the patient by the healthcare provider at the time of patient intake, in an embodiment. The disclosure, which would require acceptance by the patient, performs at least one of the following, but is not so limited: authorizes a subscriber to enter patient information into the system; authorizes a subscriber to access the system database to determine if the patient is a patient debtor.

As described in detail herein, the PDS includes a platform, application and/or database for use in the medical/healthcare industry, for example, to facilitate immediate review of a proposed patient's prior negative payment history with healthcare providers. The healthcare providers include, but are not limited to, physicians, clinics, hospitals, imaging centers, physical therapists, dentists, orthodontists, and any person or entity providing healthcare services to patients. The system provides near-immediate healthcare-related financial payment history to healthcare providers in order to help the healthcare providers facilitate a timely decision whether to treat a patient on credit, or require some form of pre-payment. Through the use of the system, each subscribing healthcare provider likely collects a large percentage of the income owed to the facility, without the need for outsourcing collection of bad debts.

The database of an embodiment does not include any identifying information relative to the nature of the medical treatment. For instance, a clinic that treats virtually all HIV or drug addicts is not made public to those who access the system database. Only the delinquent patient/guarantor can determine the identity of the creditor who is owed money. The only information in the database of an embodiment is the number of the provider, social security number of the patient/guarantor, the amount of money currently owed, and the date that this debt was introduced into the database. Date of treatment is not an issue, as a healthcare provider can choose not to put delinquent debt into a database until certain efforts of collection by healthcare provider have already been exhausted. The entry of bad debt is left to the complete discretion of the healthcare provider.

In order to use or access the services offered, the system provides a healthcare provider with two methods under which they can access the database, a billing software method and a manual method. Under both methods, the healthcare provider becomes a subscriber to the system, and is provided a subscriber number that is unique to this healthcare provider. The membership includes payment of a membership fee (e.g., annual fee, monthly fee, periodic fee, etc.) of a pre-specified amount. The healthcare provider, under an embodiment, agrees to the terms of the contract agreement via an internet contract/subscriber agreement, for example, where the contract establishes the terms, conditions and obligations of both the healthcare provider and services provided under the system.

Under the billing software model, the database information of an embodiment is accessed and updated automatically through subscriber billing software. This software provides an automated review of a proposed patient/guarantor's negative payment history at the time a newly admitting patient is screened by a healthcare provider or at any time prior to treatment of the patient. Upon entering the name, date of birth, and social security number of the patient, the system facilitates a review of the database for any current negative payment history. If a current indebtedness is identified in the database, then this information is provided to the healthcare provider and the delinquent patient/guarantor. Based on any history of delinquency, the healthcare provider then makes a decision whether it requires advance payment from the patient prior to performing any medical treatment.

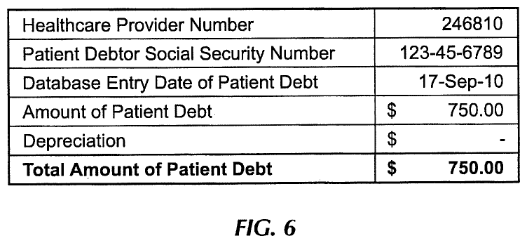

FIG. 6 is an example of patient information of the system database, under an embodiment. In this example, the patient information includes, but is not limited to, the following: a healthcare provider number (e.g., “246810”) of the treatment provider; the patient debtor Social Security number (e.g., “123-45-6789”) or other patient identification number; the data the patient information was entered into the database (e.g., “17 Sep 10”); an amount of the patient debt (e.g., “$750.00); an amount of interest accrued on the patient debt amount (e.g., none in this example); and a total amount of patient debt (e.g., the total of the patient debt and the interest accrued on the patient debt (e.g., “$750.00)).

Further, the patient/guarantor can then be provided a report or printout reflecting the nature of the negative payment entry/entries, with information on how to immediately remove this entry upon payment of the indebtedness by facilitating payment. Under an embodiment, when receiving payment from the patient for services previously rendered by a subscriber, the system makes pro-rata payments to the treating healthcare provider, less a collection fee as set out in the contract/subscriber agreement. All negative payment information is likewise automatically entered into the database, or amended in the database, via an automated information exchange between the system and each respective healthcare provider using the medical billing software described above.

For those healthcare providers who do not own, use or lease billing software which can access the database of the system, manual access to the database is enabled after becoming a subscriber, paying the membership fee, and agreeing to the contract/membership agreement terms. Under the manual method, the healthcare provider updates its financially delinquent patients within the database on a periodic basis (e.g., weekly, monthly, quarterly, etc.). The healthcare provider is provided access to the database by accessing an internet website or other electronic portal and entering a secure pass code. Upon gaining access to the system, the healthcare provider enters the name and social security number of the applicant/guarantor/patient, at which time this party's negative payment history (if any) is presented on a display. The negative payment display is then copied and provided to the guarantor/patient, along with contact information to facilitate removal of the negative entry upon payment.

The PDS of an embodiment integrates with other third party service providers involved in the billing and collection of amounts corresponding to services rendered by healthcare providers. Following are a number of examples of integration of the PDS with a third party billing company that provides billing services to providers.

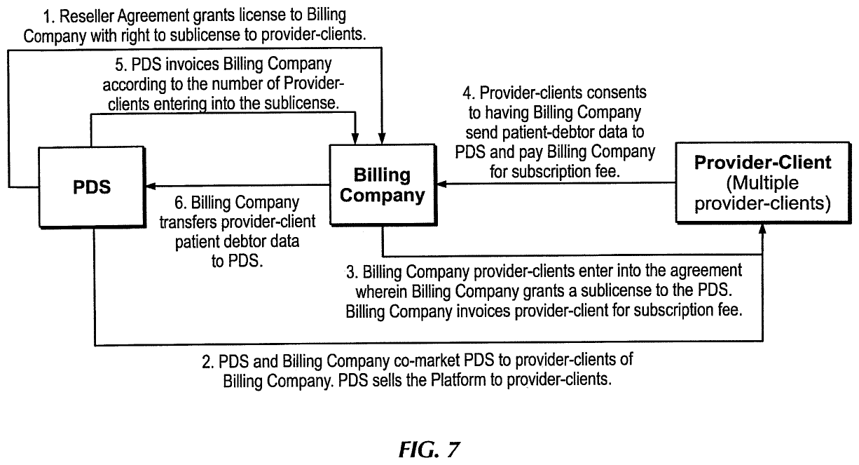

For example, FIG. 7 is a block diagram of a healthcare revenue environment in which the patient-debtor service (PDS) is provided to healthcare providers through an intermediate billing company, under an embodiment. In this embodiment, the provider accesses the PDS via an arrangement with the billing company, and the billing company receives the patient information from the provider and in turn provides the patient information or at least a subset of the patient information to the PDS. More particularly, a license is provided to the billing company with a right to sublicense to provider-clients of the billing company. The PDS and the billing company co-market the PDS to provider-clients of the billing company, and the PDS sells the platform services to provider-clients. Billing company provider-clients enter into agreement wherein the billing company grants a sublicense to the PDS, and the billing company invoices the provider-client a subscription fee. Provider-clients consent to having the billing company send patient-debtor data to PDS and pay billing company a subscription fee. The PDS invoices the billing company according to the number of provider-clients entering into the sublicense. The billing company subsequently transfers provider-client patient debtor data to the PDS.

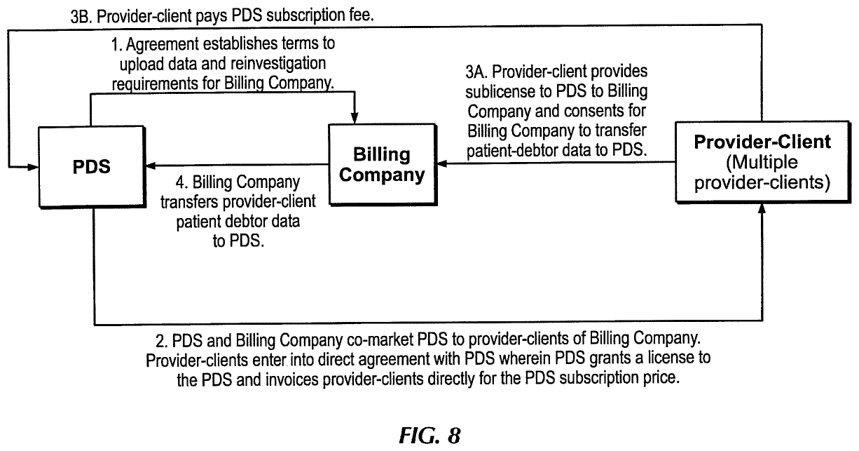

FIG. 8 is a block diagram of a healthcare revenue environment in which the patient-debtor service (PDS) is provided directly to each healthcare provider, under an alternative embodiment. In this embodiment, the provider accesses the PDS directly, and the billing company receives the patient information from the provider and in turn provides the patient information or at least a subset of the patient information to the PDS. More particularly, an agreement between PDS and the billing company establishes terms to upload data and reinvestigation requirements for the billing company. The PDS and billing company co-market the PDS service to provider-clients of the billing company. The provider-clients enter into a direct agreement with the PDS wherein the PDS grants them a license to the PDS and invoices the provider-clients directly for the PDS subscription price. In turn the provider-clients provide a sublicense to the PDS to the billing company and consent for the billing company to transfer patient-debtor data to the PDS. The billing company subsequently transfers provider-client patient debtor data to PDS.

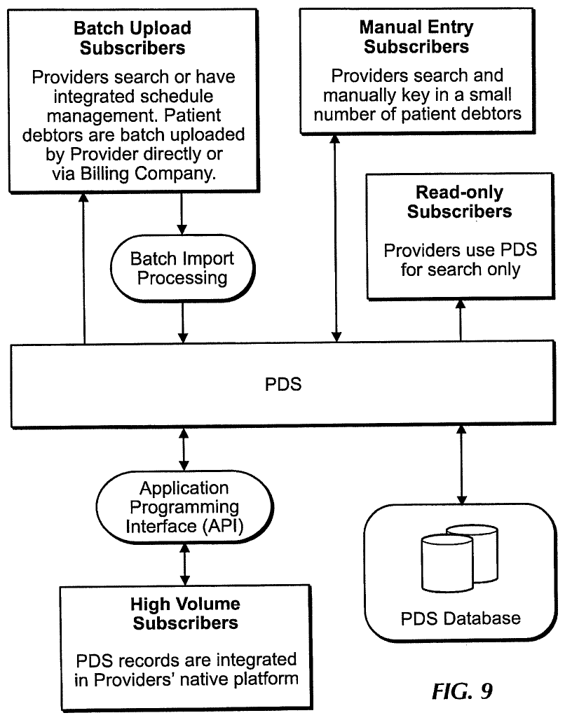

There are numerous classes of provider subscribers to the PDS of an embodiment. FIG. 9 is a block diagram showing classes of subscribers to the patient-debtor service (PDS), under an embodiment. The PDS platform of an embodiment separates subscribers into four different classes, but is not so limited. Search-Only subscribers use the PDS web application to search the database or generate reports to analyze the risk of patients before care. This class of subscriber might pay a higher price to use the service because they do not contribute delinquent debts. The Manual Entry subscriber manually keys in a small volume of patient debt and could pay a slightly lower price for this contribution. The Batch Upload subscriber has a moderate volume of patient debts that are uploaded, periodically. The batch uploading is a file-based export or report that is generally performed in-house or as part of an outsourced business process. High Volume subscribers make use of an application programming interface (API) of the PDS to exchange debt records and integrate the tracking and notification within their existing patient management platform.

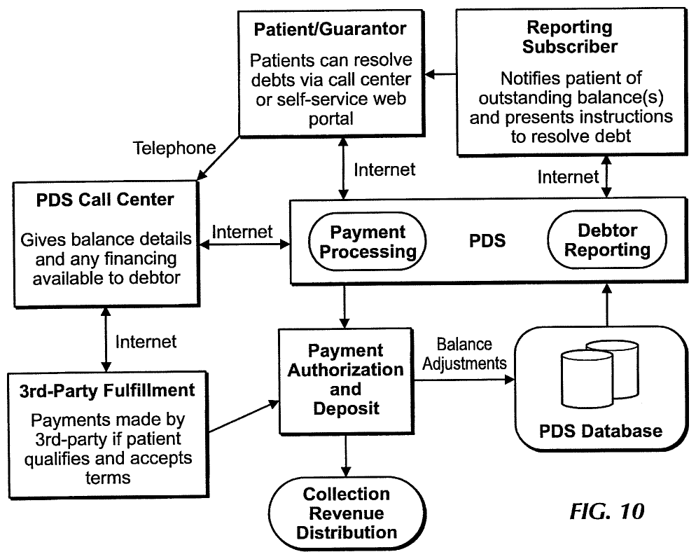

FIG. 10 describes the debt resolution process of the PDS after a subscriber notifies the patient of their delinquent status, under an embodiment. A statement of accounts is generated by the subscriber for the patient debtor, and this event is tracked in the PDS platform in order to compensate the reporting provider for acting as an agent for the provider owed in the case of collection. When notified of an outstanding debt, the patient has the option to resolve the outstanding debt at the subscriber's point of service where payment processing is accomplished through the subscriber's account on the PDS platform. Once payment is confirmed the balance is adjusted and from that point in time the other subscribers of the PDS will have no knowledge that the debt existed. The subscriber that collected the balance from the patient-debtor might be paid a commission based on the amount collected from the patient-debtor.

Embodiments also enable patient-debtors to pay outstanding debts at a subsequent time by contacting a call center or creating an account at the PDS patient access platform. Under either method, a patient can also apply for available financing to be applied to one or more patient debts. Embodiments enable simultaneous querying via the PDS of multiple financing companies in order to determine if the patient qualifies for one or more financing plans. If the patient-debtor is approved for financing, the provider will receive the negotiated amount up-front and the patient will pay installments as appropriate to the source providing the financial assistance or loan.

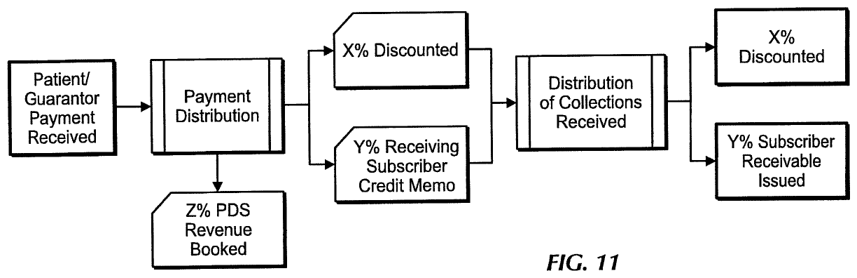

FIG. 11 is a flow diagram for distributing revenue collected via the PDS, under an embodiment. A percentage and/or transaction fee is reserved as revenue to compensate PDS under an embodiment. As an example, the last provider to report a delinquent debt to a patient could be issued a commission based on the amount collected from that patient, and the remainder of the amount collected would go to the provider that provided the services corresponding to the debt, under an embodiment.

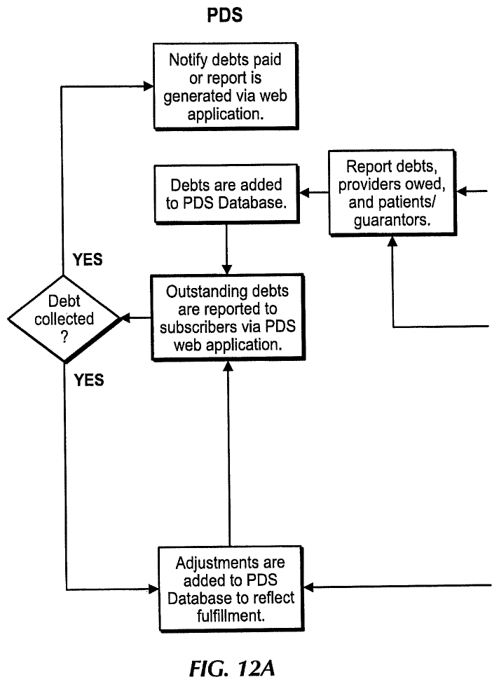

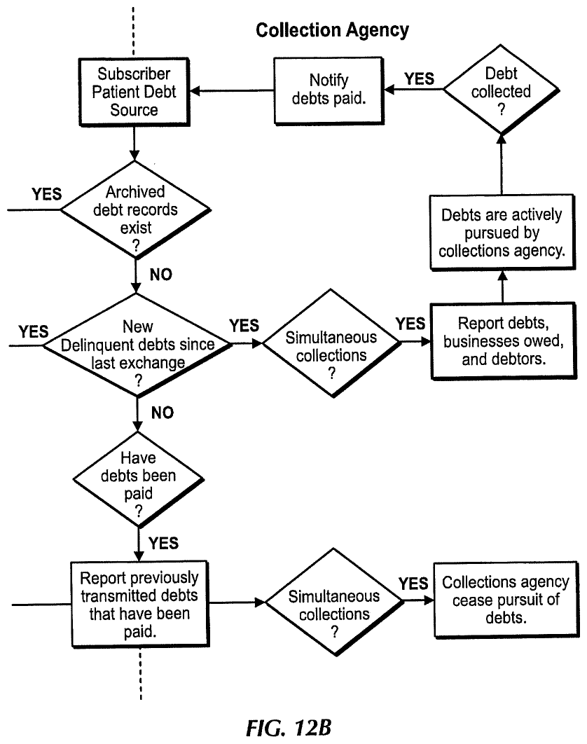

Information of aged receivables can be received and posted at the PDS platform anytime following write-off. This means the PDS service can assume the role of a primary collector, a secondary collector, or a data warehouse. Some difficulties arise however when PDS is not the sole collector of bad debt. FIGS. 12A-B are a flow diagram for collecting debts relating to healthcare services via the PDS platform and/or a collection agency, under an embodiment. As accounts are written off, they are transferred to PDS and added to the database. At the same time, accounts in this example are given to a collection agency to pursue soft and hard collections. When bad debt is settled, either party notifies the data source. Any inconsistencies are handled by the PDS platform through a dispute process, whereby the originator of the account furnishes proof to dismiss the claim. If the status of the debt cannot be confirmed within a pre-specified period of time (e.g., 30 days), the balance in question is deactivated and only reactivated once confirmed.

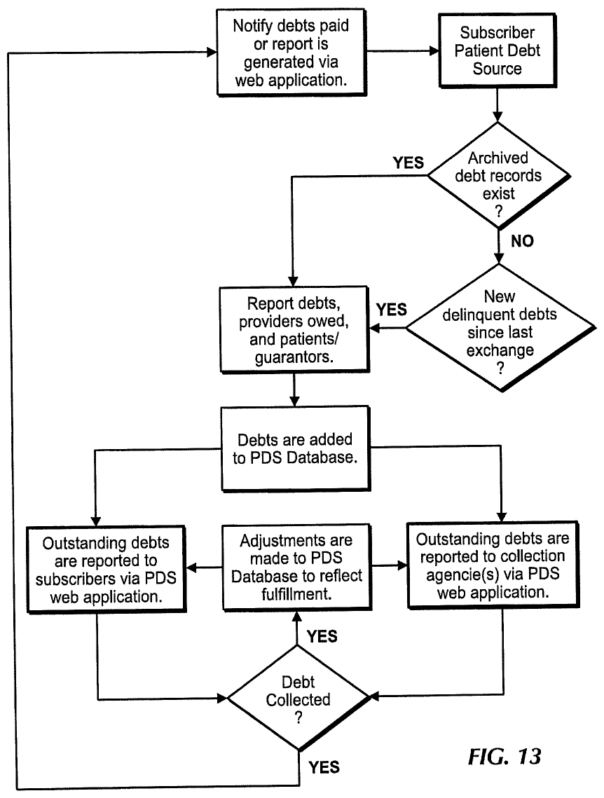

FIG. 13 is a flow diagram for collecting debts relating to healthcare services using the PDS platform as a data warehouse, under an embodiment. Under this embodiment, the PDS platform is a data warehouse or pass-through channel that communicates debts and payments to and from collections agencies. In this way, the subscriber, patient, and collection agency simultaneously receive the same account standing.

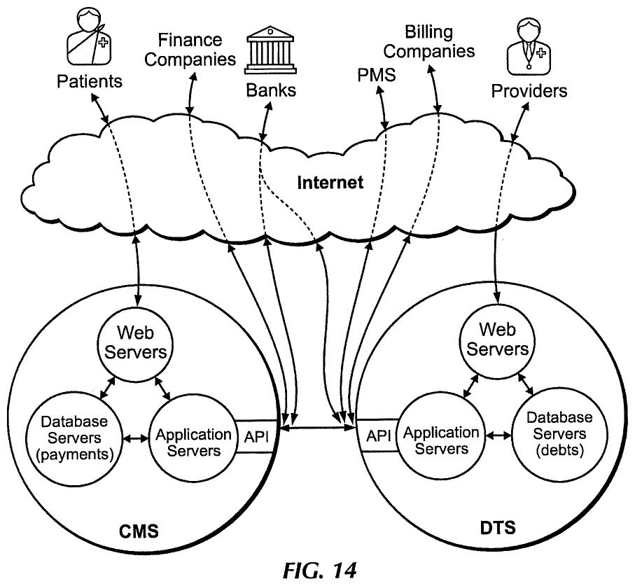

FIG. 14 is a block diagram of a patient-debtor system (PDS) that includes the patient debt tracking system (DTS) and a collection management system (CMS), under an alternative embodiment. The PDS platform includes the debt tracking and notification system described herein for tracking information of and notifying healthcare providers of financially delinquent patients. Additionally, the platform includes a collection management system that enables providers to manage collection activities relating to patient-debtor accounts.

The debt tracking system and a collection management system of the PDS of this alternative embodiment can be hosted together on a single platform, or distributed among multiple platforms and/or servers. The debt tracking system of this embodiment includes at least one web server, at least one database server, and at least one application server coupled and configured to function as described herein. In particular, subscribers of the PDS (e.g., healthcare providers, billing companies, etc.) access the debt tracking system of the PDS via a network coupling or connection (e.g., Internet, etc.) to the appropriate web server. The web servers of the PDS render user interface pages by which subscribers and other service providers access the PDS. Additionally, other third parties supporting provision of services of the PDS (e.g., billing companies, practice management systems, financial institutions, finance companies, banks, etc.) access the PDS via one or more APIs of the PDS. The APIs of an embodiment are hosted on at least one application server of the PDS but are not so limited. Subscribers accessing the PDS can access the database information directly via the database servers, or can access the information and services of the PDS via the application servers.

The collection management system is coupled and/or connected to the debt tracking system. The collection management system includes at least one web server, at least one database server, and at least one application server coupled to provide the collection management functions. In particular, patient-subscribers of the PDS access the collection management system via a network coupling or connection (e.g., Internet, etc.) to the appropriate web server of the collection management system, where the web server renders user interface pages by which subscribers and other service providers access the PDS. The other third parties supporting provision of services of the PDS as described herein access the collection management system via one or more APIs of the collection management system. The APIs of an embodiment are hosted on at least one application server of the collection management system but are not so limited. Additionally, third parties supporting the PDS can access the collection management system via web servers and/or application servers of the debt tracking system, and can access the debt tracking system via web servers and/or application servers of the collection management system. Subscribers accessing the PDS can access the database information directly via the database servers, or can access the information and services of the PDS via the application servers.

Data received at the PDS (e.g. patient data, practice data, financial data, etc.) is received from various third party data sources at the application servers of the PDS. The data sources include healthcare providers, practice management systems, billing companies, and financial institutions to name a few, but are not limited to these sources. In an embodiment the data is received at the PDS, and data transformation operations used to make any received data compatible with the PDS database are performed by at least one of the application servers.

Data exchange transactions involving the PDS include data exchanges between the PDS and a third party billing company, as described herein. The data exchanges between the PDS and the billing company include but are not limited to patient payment history balance data, data of patient/provider relationships, and service dates. The data flow is generally unidirectional except for the notification of records which fail to import. The third party billing company transfers records directly from their practice management software via a transport mechanism (e.g., HL7 MLLP protocol, etc.) or upload them through the use of the PDS portal.

Data exchange transactions involving the PDS further include data exchanges between the debt tracking system and the collection management system. For example, a patient's identity is confirmed by the collection management system via a coupling (e.g., SSL RESTful API) with the debt tracking system. The patient's balances are then transmitted from the debt tracking system to the collection management system via the same coupling (e.g., SSL RESTful API). A patient also uses the PDS to make payments or receive financing to clear balances owed to provider(s); depending on a configuration of the provider, outstanding balances could be settled in full or discounted. The collection management system reports partial payments or settled balance status to the debt tracking system via a coupling with that system (e.g., SSL RESTful API).

Data exchange transactions involving the PDS also include data exchanges between the collection management system and a non-reseller, third party billing company. The collection management system reports partial payments or settled balance status to the third party billing company via a collection management system portal or a consolidated statement. The collection management system issues payment to the third party billing company for each paid provider at agreed upon intervals.

As described in detail herein, the PDS database is an accessible database that houses delinquent patient A/R information. Healthcare providers can subscribe to the PDS to contribute patient debtor data (delinquent patient A/R data), and to access and search the patient debtor data of the PDS to determine if a patient is in the database and has outstanding debt.

If a patient is matched in the PDS and the patient desires to arrange to pay off the outstanding debt, then the patient debtor confirms the debt with the debt tracking system. In an effort to resolve the debt, an algorithm of the collection management system determines the discounted present value of the outstanding debt. For example, if the patient debtor has a $1,000 outstanding debt owed to a provider that is aged two years, then the algorithm calculates the value of the present value of the $1,000 debt, again for example, $250. Generally, the input parameters for the algorithm are derived from but not limited to data of inputted values from the provider, inputted values from the software, and parameters derived from historical data or other data sets that value an aged service receivable.

Once the value has been determined, $250 in this example, the patient determines how she/he will pay the re-valued debt. The collection management system can process a payment or payments form the patient debtor via ACH check, credit card, or other method. If the patient desires to finance the $250, the collection management system can match providers of consumer debt financing and the products these companies offer relative to the patient debtor FICO score.

The collection management system comprises a patient portal by which patient-debtors access the system. The patient can access information of their medical debt liability via the patient portal. The platform provides, via the patient portal, simple, understandable data of medical liability. A number of medical bills are sent to collections because of misinterpreted Explanation of Benefits (EOBs). For example, a patient may routinely receive EOBs showing the payer's reimbursement and co-pay tendered at time of service, which results in zero balance owed. In other cases, a small balance still exists. To the laymen, both EOBs look very similar. The patient mistakenly ignores EOBs meant to convey a balance owed because of the sheer number of EOBs sent that require no action.

In another example, the volume of medical correspondence overwhelms an ailing patient during at a time of frequent treatment. They have trouble staying abreast of the denial process or who/what they owe. Their account is charged-off and referred to a collections agency.

When accessing debt information via the patient portal, all balances are presented by the system in simple financial context as if the patient is viewing a transaction listing of a credit card or banking account. There is one balance owed per service per provider. Charges can be paid bundled or unbundled.

The patient portal of an embodiment also enables access to payment solutions. Patients can pay bills on-line in the self-serve portal with convenient methods of payment (e.g., credit card, debit, e-check) or patient financing.

The patient portal provides a fair and competitive market place empowering the consumer to expeditiously dispose of past due medical bills under a variety of payment methods. A patient can quickly determine which providers in the network they owe and resolve balances. A patient is presented with a one-time payment amount to settle all outstanding balances with provider(s) owed. This is generally the best deal. Once the transaction is complete the debt, while remaining in the database, is marked as having been paid and is thereafter never shown to providers; alternatively, the patient's debt can be cleared from the debt tracking system of the PDS.

Alternatively, an installment plan is offered by the platform without financing of payments made over the course of several months including a grace period arrangement with the debt tracking system as long as the account stays in good standing. Also included may be penalties for non-completion of a selected payment plan. Lastly, multiple finance companies can be checked concurrently and one or more used in conjunction to create a fully financed payment plan. Upon approval for financing, the patient's debt will immediately be cleared from the PDS database.

The collection management system of an embodiment also offers to settle unbundled amounts owed (one transaction fee per payment to incentivize paying everyone owed). As an example, the system may generate a greater discount on a bundled balance as greater incentive to receive payment.

The PDS of an embodiment includes a provider interface or provider portal that enables healthcare providers to manage risk using the parameters of patient debtor accounts. Using the provider portal, providers control or dictate their own aged accounts receivable (A/R) discount schedule. The discount schedule of an embodiment is generally a monotonically decreasing function representing the value of one-dollar charged-off verses time (0-2372 days or 6.5 years). By default, the discount schedule reflects a schedule that is estimated to yield the highest return based on the community aggregate for that specific provider given their local area, specialty and/or distribution of aged A/R. The built-in continuous function of an embodiment representing the debt-discounting curve can be customized by direct manipulation using a drag and drop interface. The parameters of the function are stored internally as a Bezier curve but the embodiment is not so limited. The Bezier curve is used because it requires a relatively small amount of storage and can be efficiently computed.

The PDS of an embodiment applies one discount policy to all patient debtors in order to promote fairness and standardize analytics and benchmarking. The discount schedule cannot be seen from the patient's perspective as this would allow them to forecast future values to their advantage. While most discounting schedules are monotonically decreasing, the system of an embodiment does not require them to be monotonically decreasing. For example, in a situation where a healthcare provider is in need of collecting for cash, using the PDS to temporarily vary their discount curve lower than most other providers could increase collections and result in greater practice liquidity.

For the reasons described herein, some error is acceptable when adhering to the discounting schedule of the PDS. By default, a debt can be settled if within a minimum range from the discount curve, such as a dollar increment (e.g. +/−5 dollars) or within a percentage of accuracy. This eases the burden on the PDS of computation and/or time of day accuracy, quantizes the discounting curve so the exact function is concealed from a patient even after multiple attempts to pay, randomizes quantization to further increase secrecy (to the patient debtor), and allows the collection management system bargaining room to settle debts.

The collection management system includes a reporting dashboard that is a customizable debt management dashboard. The dashboard of an embodiment provides real-time benchmarking by comparing a selected discount schedule to an average local aggregated discount schedule. The dashboard also generates and provides to subscribers a number of reports, including but not limited to A/R aging schedules, average A/R days, and collection ratio (Percent of Accounts Receivable Beyond 60, 90, and 120 Days (PARB60, PARB90, and PARB120)).

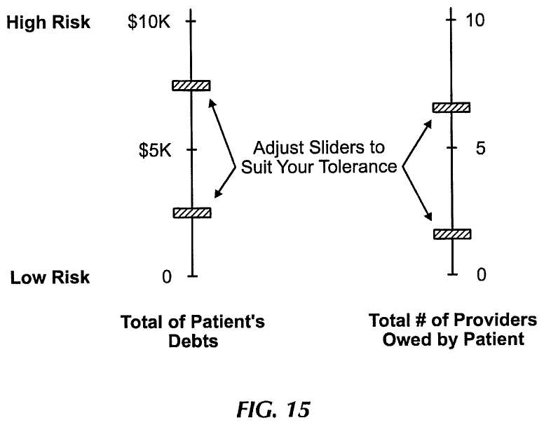

The provider portal of the PDS enables healthcare providers to manage risk using the parameters of patient debtor accounts as described in detail herein. This interface is accessed via one or more of the debt tracking system and the collection management system. The interface includes one or more controls or sets of controls, and each control corresponds to a risk or account parameter that can be adjusted or selected by the healthcare provider. FIG. 15 is an example user interface including threshold-setting controls for collection management parameters, under an embodiment. This example interface includes a first set of adjustable parameters corresponding to amounts owed to the healthcare provider by patient debtors, and a second set of adjustable parameters corresponding to a total number of healthcare providers owed by patient debtors. In this example embodiment, a healthcare provider uses the first set of adjustable parameters to set a “Low Risk” tolerance by setting a first control to an amount that is the maximum threshold amount of patient debt considered as low risk; this first control also establishes the amount that is the minimum threshold amount of patient debt considered as medium risk. The healthcare provider also uses the first set of adjustable parameters to set a “High Risk” tolerance by setting a second control to an amount that is the minimum threshold amount of patient debt considered as high risk; this second control also establishes the amount that is the maximum threshold amount of patient debt considered as medium risk.

Continuing the example, the second set of adjustable parameters corresponds to a total number of healthcare providers owed by patient debtors. A healthcare provider uses the second set of adjustable parameters to set a “Low Risk” tolerance by setting a first control to a number that is the maximum threshold number of providers owed by the patient debtor considered as low risk; this first control also establishes the number that is the minimum threshold number of providers owed by the patient debtor considered as medium risk. The healthcare provider also uses the second set of adjustable parameters to set a “High Risk” tolerance by setting a second control to a number that is the minimum number of providers owed by the patient debtor considered as high risk; this second control also establishes the number that is the maximum threshold number of providers owed by the patient debtor considered as medium risk.

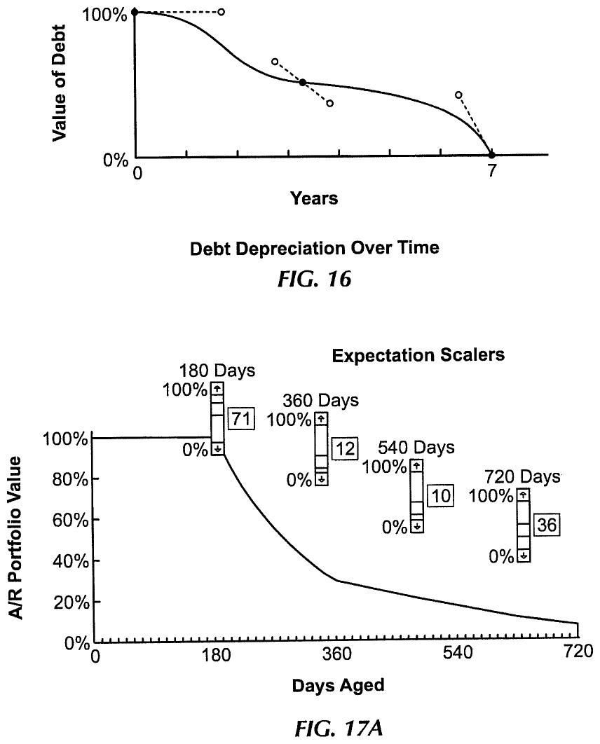

FIG. 16 is an example user interface including debt depreciation controls for collection management parameters, under an embodiment. Past due accounts translate into major dollar losses. Foe example, healthcare providers are often the last to be paid. A $500 account 30 days past due is typically worth only $440, and that same account 120 days past due might be worth only $260. Using this example, the PDS includes an interface that enables healthcare providers via the collection management system or component of the PDS to manage collections from patient debtors and manage depreciation (or revaluation) of debt over time. This example interface includes an adjustable debt depreciation curve by which the provider can set any number of points on the curve. In this manner the provider customizes and shapes their debt depreciation curve and in so doing manages collections from patient debtors according to age of the debt.

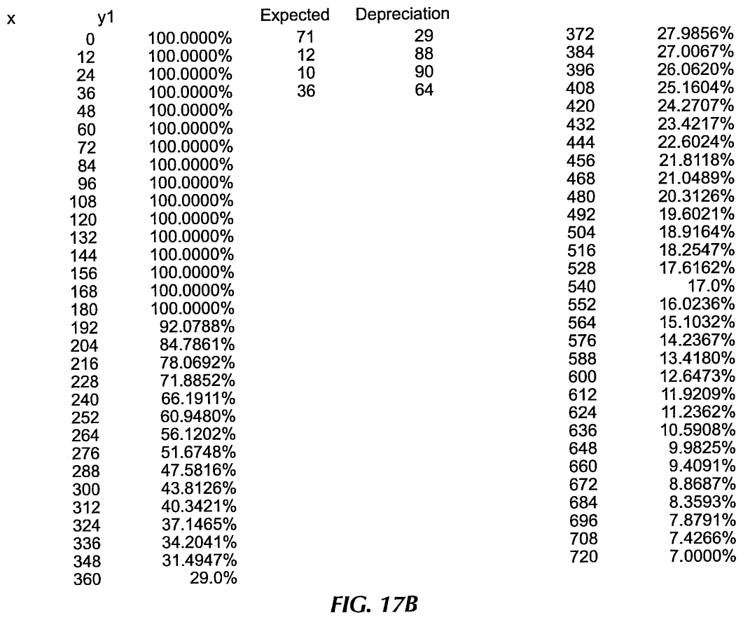

FIGS. 17A-B are another example user interface including debt depreciation controls for collection management parameters, under an embodiment. In this example, the PDS enables a provider to customize their debt depreciation using one or more controls, each of which corresponds to an age range of the debt. As a particular example, the interface includes a first control that allows a provider to select a collection percentage for debt having an age greater than zero (0) days and up to 180 days, a second control that allows a provider to select a collection percentage for debt having an age greater than 180 days and up to 360 days, a third control that allows a provider to select a collection percentage for debt having an age greater than 360 days and up to 540 days, and a fourth control that allows a provider to select a collection percentage for debt having an age greater than 540 days and up to 720 days. In this manner the provider controls the debt depreciation and in so doing manages collections from patient debtors according to age of the debt.

The PDS of an embodiment includes one or more interfaces that enable providers to control collection activities on patient-debtor accounts. Additionally, the PDS of an embodiment automatically provides recommendations of collection management parameters for selection by a healthcare provider via the PDS interface. The recommendations can be based on any number or combination of parameters (e.g., practice type, practice size, practice specialty, popular selections of other providers, etc.) and are generated to provide optimal return.

The PDS of an embodiment is configured to couple with and use information of the HIPAA Eligibility Transaction System (HETS). The HIPAA Eligibility Transaction System (HETS) is intended to allow the release of eligibility data to Medicare providers, suppliers, or their authorized billing agents for the purpose of preparing an accurate Medicare claim, determining beneficiary liability or determining eligibility for specific services. In this environment, the healthcare provider (submitter) transmits a 270 eligibility request file. A response file or form is returned in response to filing of the 270 request. The 270 request and the 271 response are “paired” transactions to determine patient insurance eligibility. The 270/271 process operates in a real-time environment such that healthcare providers and clearinghouses may initiate a real-time 270 eligibility request to query coverage information from payers (insurance companies) on patients for whom services are scheduled or have already been delivered. In the real-time mode, a healthcare provider or clearinghouse transmits a 270 request and remains connected while the receiver processes the transaction and returns a 271 response.

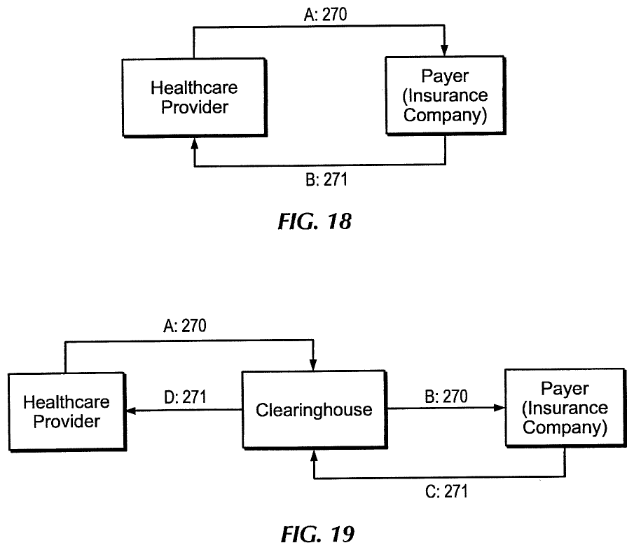

FIG. 18 is a flow diagram between a healthcare provider and the payer for the 270/271 transaction. The 270 request and the 271 response are “paired” transactions as described herein. The 270 request is an inbound insurance eligibility request sent from a provider, and the 271 is an outbound insurance eligibility response sent to the provider.

FIG. 19 is a flow diagram of the 270/271 process between the healthcare provider and the payer via a clearinghouse. Again, as described herein, the 270 request (inbound insurance request) and the 271 response are “paired” transactions, however under this process the 270 is sent from the healthcare provider through a clearinghouse to the payer. There can be more than one clearinghouse in the connection.

The PDS of an embodiment aggregates patient-debtor data from healthcare providers. Subscribing healthcare providers can access the PDS via an electronic inquiry to determine whether a patient is in the PDS database. Generally, the inquiry comprises the patient's name, address, social security number (SSN), and date of birth, as described herein, but is not so limited. The PDS inquiry submitted by the provider is similar to the patient information of the 270/271 insurance eligibility.

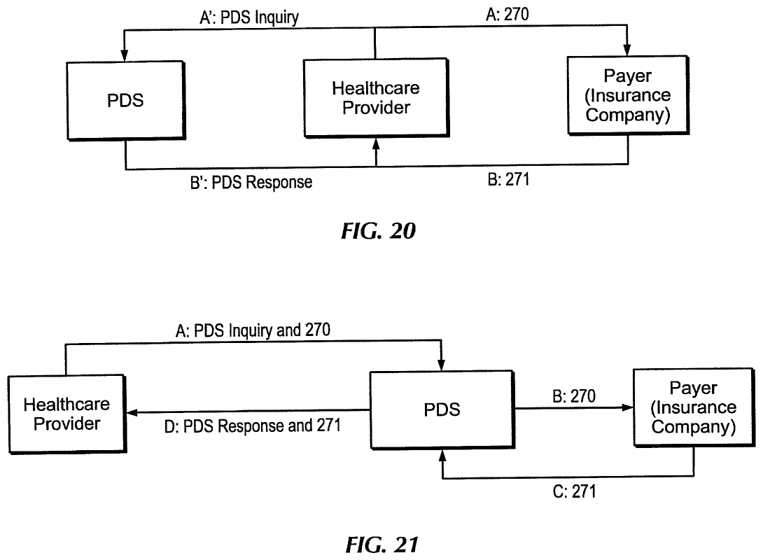

FIG. 20 is a block diagram of the PDS coupled with a 270/271 HETS process in which the PDS and the payer transact with the 270/271, under an embodiment. The 270/271 process is bifurcated when it is sent form the healthcare providers practice management system (PMS), because the 270 request is separately routed to the PDS and to the payer (e.g., A′ and A respectively). Both the PDS and the payer return a response (B′ and B respectively).

FIG. 21 is a block diagram of the PDS coupled with a 270/271 HETS process in which the PDS is an intermediary in the 270/271 process, under an embodiment. The combined PDS-270 inquiry is sent form the healthcare provider's PMS (A) and is received first by the PDS. The 270 passes through the PDS to the payer (B) and the PDS receives the 271 response (C). The combined 271 and PDS response is sent to the healthcare provider (D). Alternatively, the PDS response and 271 do not have to be combined and can be sent sequentially when the inquiry has been process either by the PDS or the payer.

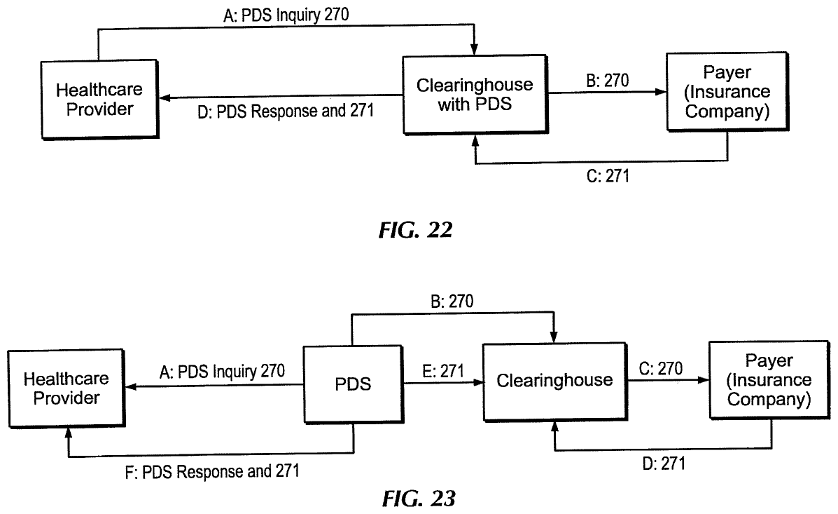

FIG. 22 is a block diagram of the PDS coupled with a 270/271 HETS process in which the PDS is integrated into a clearinghouse that intermediates the combined PDS inquiry and 270 request, under an embodiment. The PDS is integrated into the processes of a clearinghouse for healthcare transactions such as Emdeon for example. The combined PDS-270 inquiry is sent from the healthcare provider's PMS (A) and is received first by clearinghouse with the integrated PDS. The 270 passes through the clearinghouse to the payer (B) and the clearinghouse-PDS receives the 271 response (C). The combined 271 and PDS response is sent to the provider (D). Alternatively, the clearinghouse-PDS response and 271 do not have to be combined and can be sent sequentially when the inquiry has been process either by the clearinghouse-PDS or the payer.

FIG. 23 is a block diagram of the PDS coupled with a 270/271 HETS process in which the PDS and a clearinghouse separately intermediate the combined PDS inquiry and 270 request, under an embodiment. The healthcare provider of this embodiment sends the PPS-270 (A) inquiry from the provider's PMS that is received first by the PDS. The 270 is passed through the PDS and to the clearinghouse (B), which subsequently sends the 270 to the payer (C). The payer returns a 271 response to the clearinghouse (D), and the 271 response is subsequently sent to the PDS (E). A combined PDS response and 271 response can be sent to the healthcare provider (F) or, alternatively, the response or PDS response can be sent when processed.

With declining insurance reimbursement coupled with increasing patient co-pay and/or self-pay responsibilities, healthcare providers such as physicians and hospitals are faced with increasing patient receivables including delinquent patient accounts receivable (A/R) and bad debt. The PDS of an embodiment includes methods and devices to assign credit worthiness of the patient and/or provider patient population and determine a score that includes but is not limited to a combination of a number of elements such as credit history using traditional FICO scoring from the larger credit reporting agencies, employment history, rental history, age, and positive credit history in the PDS database.

The PDS of an embodiment determines an aggregate scalar by scoring a healthcare providers patient demographic as well as the provider's patient accounts receivable history associated with the demographic. In particular, the proposed embodiments determine, for example, the Provider Patient Credit Metric (PPCM) score, which is an aggregate score for the healthcare provider's patient or patient population.

For example, the PPCM score can be used to determine the credit worthiness of a provider's patient population and the aggregate risk this population poses to the provider's A/R. The PPCM score can range from 0 to 1,000, with 0 reflecting the greatest risk and highest likelihood a patient will not pay for healthcare services to 1,000 reflecting a perfect score (no-risk) and a near perfect propensity to pay for healthcare services. A score of 500 is deemed to be average while scores of 250 and 750 are below and above average, respectively.

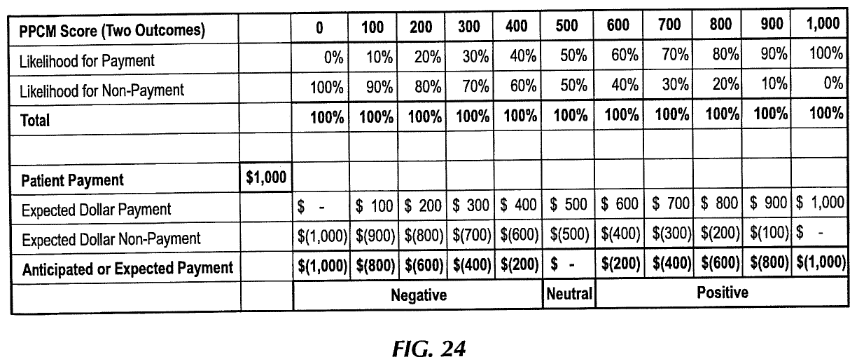

FIG. 24 shows data of an example scenario for the PPCM score and expected dollar payment from a patient, under an embodiment. This example presents PPCM scores for two outcomes (likelihood for payment or likelihood for non-payment) from out-of-pocket patient payment for healthcare services. A PPCM score from 0 to 499 is considered below average with a negative dollar expectation value (provider would expect no payment). A PPCM value of 500 is considered neutral or zero dollar expectation value. A PPCM value from 501 to 1,000 is considered above average with a positive dollar expectation value (provider would expect payment).

Similarly, a healthcare provider's A/R can also be scored and relative to the provider's PPCM score. For example, one would expect a provider's having a PPCM score of 250 to have large patient accounts receivable coupled of significant aged receivables and bad debt. Furthermore, it would be expected that the occurrence of patient debtors in the PDS would be high when compared to a healthcare provider with the PPCM score of 750.

From the PPCM scores and other parameters and measures, self-insured and/or institutional insurance policies that cover bad debt and/or purchase aged receivables can be formulated and implemented into the payment of healthcare services. For example, if a patient has a low FICO score and/or appears in the PDS database, then a premium can be charged or set aside at the time healthcare services are delivered. If the healthcare service charge to the patient is $100, for example, and the patient pays $50 at the time services were delivered, then $10 would be charged as a premium. As such, the patient's outstanding balance would be $60. If the patient pays the amount in full, then either the $10 is reimbursed or credited towards the patient balance.

A provider can insure his/her receivables given the PPCM score of the provider's aggregate patient demographic. The lower the PPCM score, the greater the risk and corresponding premium.

The PDS of an embodiment comprises components for financing healthcare treatment that include an electronic infrastructure that automatically retrieves patient data from the PDS database and/or a patient management system of a service provider. A credit application is generated by automatically populating a credit application form of the patient financing system using the patient data. Custom credit applications are generated by mapping the credit application to the custom credit applications, with each custom credit application corresponding to one of a number of credit providers. The custom credit applications are electronically submitted to the credit providers. Electronic credit decisions are received from the credit providers in response to submission of the custom credit applications. A decision on a selected credit provider is received and, in response, an electronic acceptance form is generated and submitted to the selected credit provider. Notification of the decision is sent to the credit providers not selected.

Moreover, systems and methods for managing healthcare service provider accounts receivable relative to insurance revenue cycles and/or consumer debt revenue cycles are described below. The systems and methods include, in an embodiment, an electronic infrastructure of the PDS for managing the revenue cycle of healthcare providers. The provider revenue cycle enabled by the PDS of an embodiment comprises a direct insurance revenue cycle (DIRC), a direct consumer debt revenue cycle (DCDRC or Patient Financing System (PFS)), and a combined provider insurance and consumer debt revenue cycle, each of which is described below. The systems and methods described herein include and/or run under and/or in association with a processing system embodied in the PDS, the provider finance platform, the DIRC and DCDRC.

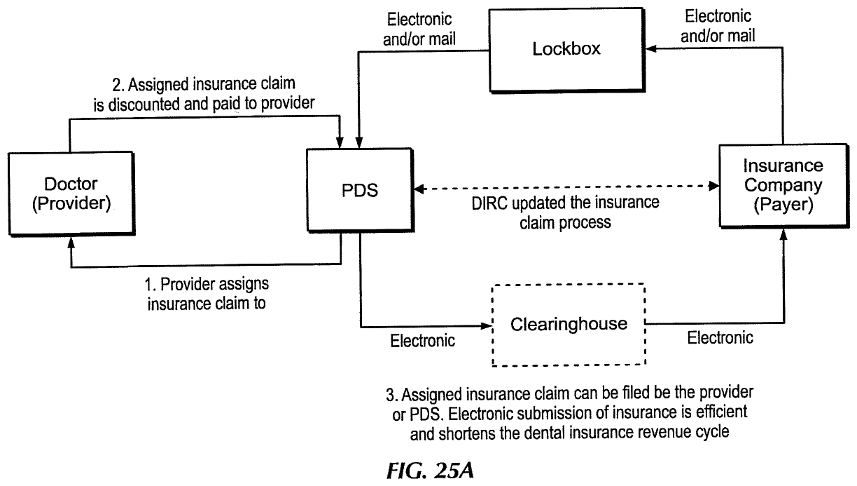

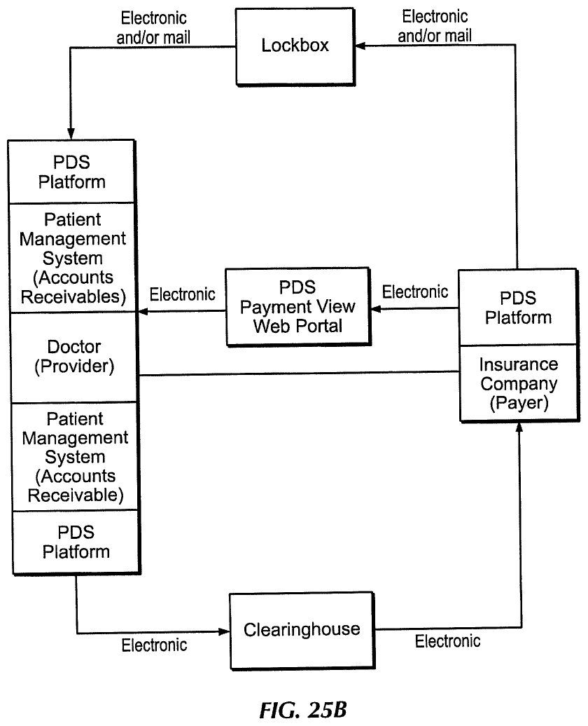

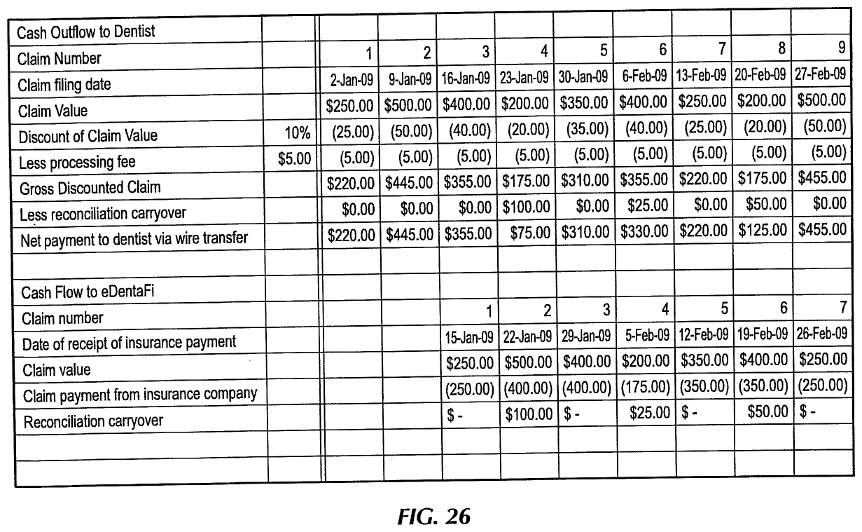

FIG. 25A is a flow diagram of the DIRC enabled by the PDS, under an embodiment. FIG. 25B is a block diagram of the system or platform components for the DIRC, under an embodiment. FIG. 26 is an example schedule of DIRC account reconciliation for automated or selected factoring, under an embodiment. The platform, including DIRC (with and without factoring), is electronically coupled to one or more computers or systems (e.g., patient management system) at a treatment facility (e.g., doctor's office, hospital, etc.) and to insurance companies or payers. The coupling includes any type or combination of network technologies as described herein. With reference to FIGS. 25A and 25B the elements of the DIRC include, but are not limited to scoring, auto-reconciliation, factoring, and insurance revenue recovery, each of which is described below.

The scoring of an embodiment is a measure of the provider insurance reimbursement process at the level of the provider practice which incorporates the following but not limited to: patient population and their insurance carrier (private versus public, mix); provider office processes used for provider insurance reimbursement (paper versus electronic); current and historical aged insurance receivable (which is a indirect measure of the insurance reimbursement process adopted by the provider); type of provider practice, i.e., solo versus group, specialty, location (urban versus rural), etc.; and, number of provider claims submitted and insurance amounts reimbursed. The systems and methods herein are not limited to provider applications as they can be implemented with many types of service providers.

The factoring of an embodiment includes automated factoring and selective factoring. The insurance revenue recovery of an embodiment comprises automated insurance recovery and selective insurance recovery.

A host provider office integrates the DIRC platform into the provider practice using the platform. The integration is accomplished via a subscription agreement (monthly fee) and/or per-claim fee (per transaction basis), for example. The DIRC can run or be accessed in the background of provider practice management systems, via a web portal, and/or as a stand-alone offering.

A host provider office implementation of the DIRC begins with a patient completing patient information forms that collect the patient name and address as well as their provider insurance profile. The patient is examined by the provider, and a treatment plan is presented to the patient. For some payers, a pre-determination (or eligibility) of provider insurance benefits and the amounts paid can be procured before the delivery of provider treatment. Provider treatment is delivered and the provider claim is submitted for adjudication and payment by the patient's insurance company.

The provider insurance revenue cycle generally comprises one or more of the following:
1. The provider assembling a bill or claim describing the services that the provider provided to the patient.
2. The accounting for the provider services is entered into the provider's practice management system.
3. The provider submits the claim to the insurance company via an electronic submission, facsimile, or U.S. mail.
4. The insurance company receives and reviews the claim against a contract (insurance policy) to determine the amount, if any, to be paid by the insurance company for the claim (services provider by the provider) to the patient.
5. The insurance company mails a check or sends a electronic funds transfer (EFT) to the provider for the claim amount and send a detailed description of the services that were paid to the provider (referred to as an Explanation of Payment or EOP and can be sent electronically in the form of an electronic remittance advice or ERA) and a similar description of services sent to the patient detailing payments to the providers (referred to as an Explanation of Benefits or EOB).
6. In the accounting module of the practice management system, the provider manually matches the provider insurance payment with the provider, claim, and balances and reconciles the payment to update the patient's account.

Insurance payments and accompanying EOP corresponding to a claim can be sent directly to the provider or the provider's bank via a lockbox. The lockbox is a facility comprises a post office box that is monitored by the bank or lockbox facility, for example. The lockbox mail generally comprises paper checks and remittance advices from provider insurance companies, which is opened by the lockbox facility. Insurance information is digitally scanned by the lockbox facility with the information forwarded to the provider and checks deposited into the provider's bank. The provider insurance company may send ERA directly to the provider or to a clearinghouse for distribution to the provider.

The provider manually posts the insurance payment and payment information into their practice management system, and in particular, the patient's account receivable file. This is a labor-intensive process that is susceptible to human error and theft. The payment information is posted and matched to the claim information. The patient's account is balanced and reconciled, and the patient is responsible for any outstanding unpaid balance.

A clearinghouse serves several purposes including but not limited to helping the provider route provider claims to various provider insurance companies through on electronic connection for example. Clearinghouses may also assist provider insurance companies by consolidating claims from many different providers. Clearinghouses may, at times, edit a provider claim to enable the provider insurance company to process the claim to their claim format.

The provider insurance revenue cycle is complex and fragmented. Providers are disadvantaged and challenged to navigate the provider insurance claims process with the myriad of provider insurance plans and provider insurance companies, many times each with their own proprietary claim requirements, adjudication and reimbursement procedures. Providers are compelled to comply with extensive federal and state laws and regulations such as the Health Insurance Portability and Accountability Act of 1996 (HIPAA) and federally mandated reimbursement procedures. At the time provider services are delivered and covered in some part by provider insurance, providers may collect a portion of the amount due from the patient and/or defer collection from the patient after payment is received from the provider insurance company, at which time the patient's account receivable balance is reconciled.

With reference to FIGS. 25A and 25B, following is a description of integration of the DIRC into a provider practice and provider revenue cycle when factoring a claim. To factor a provider claim under the embodiments herein, the platform intermediates the provider insurance revenue cycle and improves the provider insurance revenue cycle and significantly reduces the provider's insurance accounts receivable balance. Since the platform streamlines and optimizes the provider insurance revenue cycle, the provider is advantaged. In addition, when factoring provider insurance claims, the provider significantly reduces their provider insurance accounts receivable. The provider claim is assigned to the platform service provider through the DIRC platform. Assignment of the claim can be automatic if the provider has subscribed for automated DIRC or selective allowing the provider to select certain patients or insurance policies to be processed using DIRC.

The assigned claim is discounted and paid. In particular, the provider insurance claim is assigned to the platform provider and the platform discounts the claim value and pays the provider a portion (discount) of the claim value (typically on the order of 75% to 80% of the claim value). The discount factor is predetermined and can be tied to the scoring of the provider office (e.g., patient population cross section of carriers, etc), type of insurance policy, procedure, and amount. The amount of the provider claim is discounted appropriately and the discounted amount is paid to the provider within a pre-specified period of time (e.g., 24 hours). The claim assignment is automatic or selective. Automatic assignment is executed under an agreement to automatically assign provider insurance claims to the DIRC for factoring and processing. Selective assignment is non-automatic and the provider office can manually select, through the platform web portal, whether to assign and factor a particular claim.

In FIG. 25B, the provider insurance claim enters the insurance claim processing side of the electronic DIRC. The provider claim is generally submitted electronically via a provider clearinghouse (e.g., ProviderXchange), but is not so limited. The provider claim is adjudicated and paid by the payer (provider insurance plan or provider insurance company). The amount of the claim paid by the payer or insurance company is collected by the platform. The payment in the form of a check and supporting detailed information about the payment can be sent via U.S. mail to the lockbox. The lockbox facility opens the mail, digitally scans the insurance check and supporting payment information, and electronically sends the information to the platform. This information is electronically imported by the platform and posted to the payee's (assigned claim) account in the platform. Alternatively, the payment can be sent electronically and the payment electronically posted to the payee's accounts in the platform. After the insurance payment is matched against the assigned claim, it is reconciled and posted. Any unpaid is amount is taken against future discounted or factored provider insurance claims by the provider or reconciled in a timely manner with the provider (see FIG. 26 for an example reconciliation for a provider that is a dentist). Alternatively, the claim can be re-submitted with or without additional information for review and possible payment by the payer.

In considering automatic and selective factoring and account reconciliation, in the patient provider billing cycle, if the patient has provider insurance the process of initiating a provider insurance claim with the intent to factor brings forward the DIRC platform to integrate the provider claim, payment of provider claim by payer, and factoring process. Once the provider claim is assigned to the platform, the platform will intermediate and link the provider insurance revenue cycle transacted through the platform to the provider's practice management system. The DIRC is optimized to file the claim electronically or can be integrated to allow the provider's resident practice management system to process and file the assigned provider claim. The provider insurance claim form is populated with provider services delivered as well as the patient's name, address, provider insurance coverage, provider delivery the provider services. If necessary, a radiograph (digital or film) or other supporting data is attached to the provider insurance claim or any other aspect of the claim necessary to show that treatment has been delivered. Once the claim has been assigned to the platform service provider and submitted to the payer (provider insurance company), the claim value is discounted and payment in the form of a wire transfer or paper check is sent to the provider within a pre-specified period (e.g., 24 hours) less any other reconciled amount (see FIG. 26) such as a processing fee. Upon assignment of the provider claim to the platform and transfer of discounted cash proceeds for the discounted claim, the platform would send a file to auto post or manually post the transaction to the provider's practice management system at the level of the patient account.

The DIRC electronically tracks the assigned provider claim during the adjudication process and can update the provider as to whether the claim was paid in full, partial payment was made and the reason for the partial payment, or the claim was denied and the supporting documentation. The provider can log into their platform account via the platform web portal and can track the progress of their assigned claim or the status of their account. If the claim was partially paid or denied, the DIRC can send a message to the provider office noticing the provider practice that the claim will be re-submitted in an attempt to overcome the denial or partial payment (see FIG. 25A), or not re-submitted and reconciled against the current reconciliation carryover (see FIG. 26). During the re-submission process, DIRC reconciles the difference with the next claim submitted by the provider office (see FIG. 26).

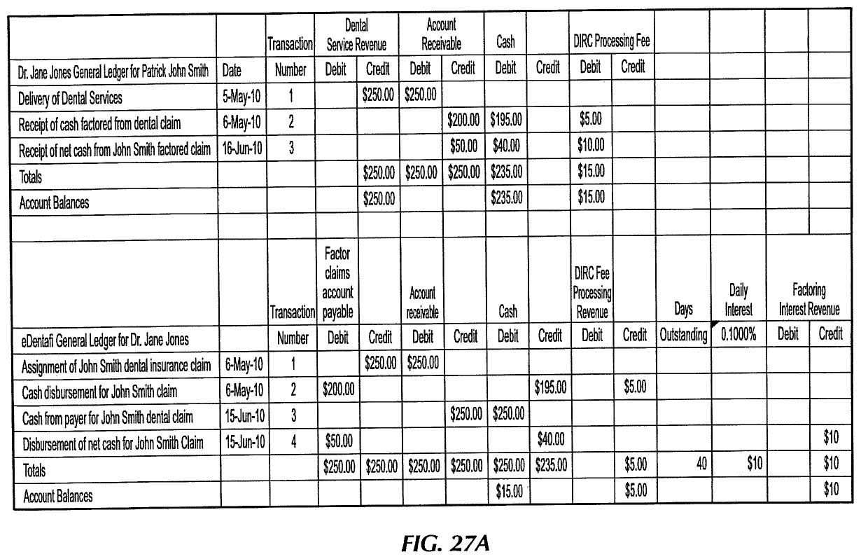

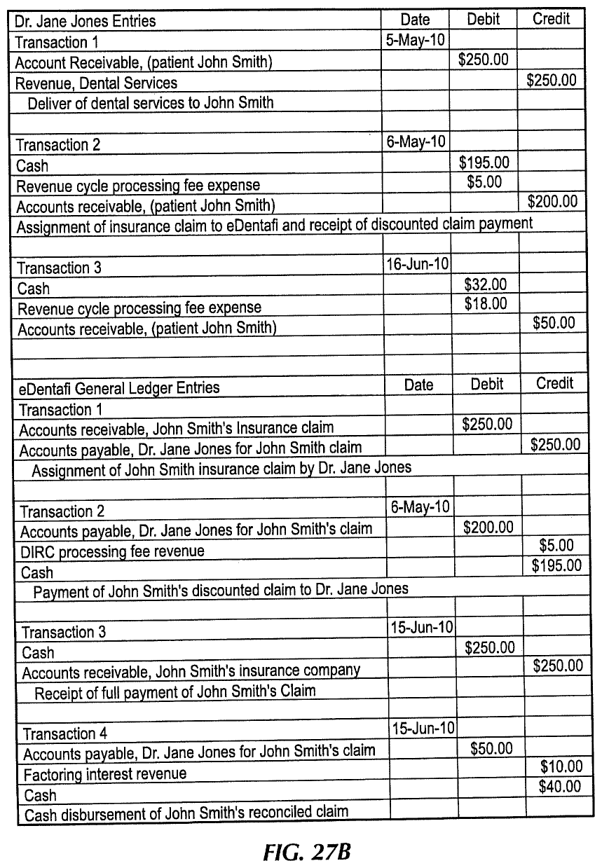

FIGS. 27A-B shows a more detailed transaction summary of an example assignment of John Smith's provider claim to the platform by Dr. Jane Jones (provider-dentist) after she delivered $250.00 in provider treatment to Mr. Smith on May 5, 2010, under an embodiment. More specifically, this example tracks accounting transactional information that is exchanged between the platform and the provider's practice management system at the level of the patient account. Furthermore, this information from the platform can be automatically or manually posted to the patient's account. If automatically posted, the provider logs into the platform and the patient account information is downloaded into the provider's practice management system at the level of patient accounting. The claim was assigned to the platform service provider via the platform web portal and the claim electronically submitted to Mr. Smith's provider insurance plan on May 5, 2010 by Dr. Jones' practice management system. On May 6, 2010, the platform accepted the assigned provider claim with a value of $250.00. Alternatively, the assigned claim could be electronically submitted by the platform to the patient's provider insurance plan. The claim was discounted 20% less a DIRC processing fee of $5.00 and an electronic wire transfer of $195.00 was sent on May 6, 2010 to Dr. Jones along with documentation for the assignment, description of payment, and postable data file for the provider's practice management system at the level of the patient account. In this series of transactions, Dr. Jones recognizes $250.00 in revenue from the delivery of provider services to Mr. Smith. Payment by Mr. Smith to Dr. Jones for provider services was deferred pending submission and payment of the $250.00 provider claim, and as such, Dr. Jones' accounts receivable for Mr. Smith shows a balance of $250.00 in anticipation of collecting some or all of the provider claim from Mr. Smith's provider insurance plan. Upon acceptance of the assigned claim by the platform, a payment of $195.00 was wire transferred to Dr. Jones along with supporting documentation. The PDS payment transaction can be sent electronically to Dr. Jones and autopost to Dr. Jones' accounts receivable module in her practice management system or her office can download the file from her platform service provider account on the platform's secure web portal. In the platform, the accounting shows an account payable to Dr. Jones of $250.00 for the acceptance of Mr. Smith's assigned provider claim and an account receivable balance from Mr. Smith's provider insurance provider plan of $250.00. After discounting the claim and deducting the DIRC processing fee, the $195.00 payment sent to Dr. Jones would reduce the accounts payable balance by $200.00 (20% discount of the claim value) and show DIRC processing fee revenue of $5.00 and cash outflow to Dr. Jones of $195.00.

After review of the Mr. Smith's provider claim by his insurance plan, the insurance company paid the $250.00 claim in full. Payment and supporting payment information was sent by U.S. mail and received by the PDS's bank lockbox. The lockbox facility opens the mail from the provider insurance plan and digitally scans the insurance payment and information. The check was deposited into the platform service provider's bank account and the payment and insurance information electronically sent to and processed by the platform. Specifically, the claim payment was matched to the assigned claim, balanced and posted to the accounts receivable module in the platform and reconciled. Specifically, the $250.00 payment received from the provider insurance plan was matched, balance, reconciled, and auto posted to Dr. Jones' account in the platform with the following results. Cash of $250.00 was debited to the platform account on Jul. 16, 2010 and Mr. Smith's provider insurance accounts receivable was credited $250.00. The provider claim was paid in 40 days and at an annual factoring rate of 36%, the platform factoring fee was $10.00, and as such, cash disbursed to Dr. Jones was $40.00.

On Jun. 16, 2010, Dr. Jones received a $40.00 cash disbursement from the platform in the form of a wire transfer for the remaining amount due from factoring Mr. Smith's provider insurance claim less the factoring fee. The payment was sent electronically to Dr. Jones and can also be sent in the form of a check and U.S. mail. The insurance information as well as the PDS file to autopost into Dr. Jones' accounts receivable module of her practice management system to reconcile Mr. Smiths account balance is either sent electronically or can be retrieved from her account on the secure the platform web portal.

In summary, Dr. Jones assigns Mr. Smith's $250.00 provider claim to be electronically factored. Dr. Jones received $195.00 of the provider claim within 24 hours of assigning the claim to the platform and electronically submitting the provider claim to Mr. Smith's provider insurance plan. Frequently, the inefficiency of provider offices coupled with the complexities of provider insurance claim submission results in aging of provider insurance accounts receivables for periods of 60 days and longer. With the platform factoring and DIRC platform, providers can receive approximately 80% of their claim within 24 hours of assigning the provider claim as well as an autopostable file showing the assignment and transaction accounting. The platform factoring significantly reduces provider's accounts receivable, which is a key financial metric to measure and manage cash in a provider practice.

Returning to the summary, upon receipt of the $250.00 payment from Mr. Smith's insurance plan, Dr. Jones' account is updated and the remaining payment electronically sent to Dr. Jones along with the platform autopost file to reconcile Mr. Smith's account in Dr. Jones' practice management system. One of the salient features of the platform centers on streamlining the insurance revenue cycle using the platform electronic insurance revenue cycle simultaneous to reducing provider's accounts receivable balances and aging. In addition, provider offices can leverage the platform to improve their provider insurance business process and streamline provider office finance and accounting processes. For Mr. Smith's $250.00 provider claim, the provider office spent a minimal amount of time managing the transaction and received approximately 80% of the provider claim within 24 hours of delivery provider services. Under the conventional provider-insurance revenue cycle commonly used by provider, the provider claim may have taken 60 days or longer to receive payment by the provider insurance company for some or all of the provider claim in addition to the significant time, resources, and overhead incurred by the provider office to manage the provider insurance revenue cycle.

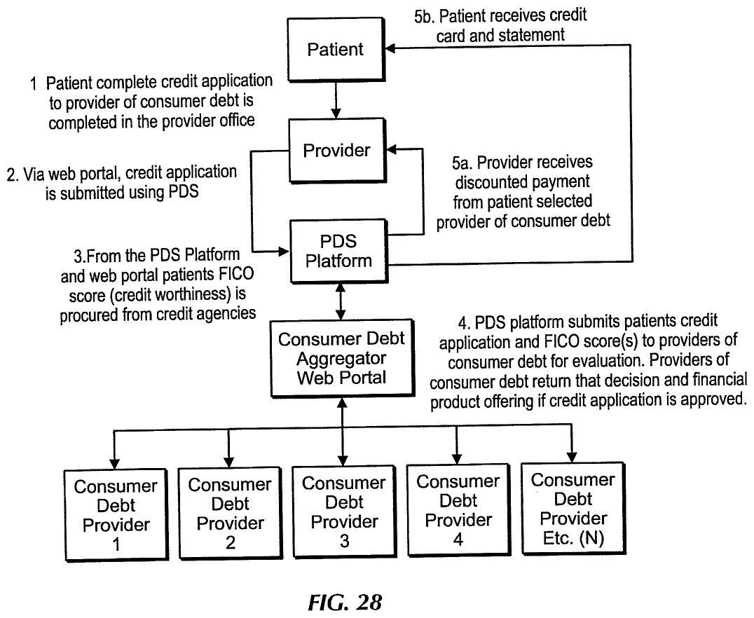

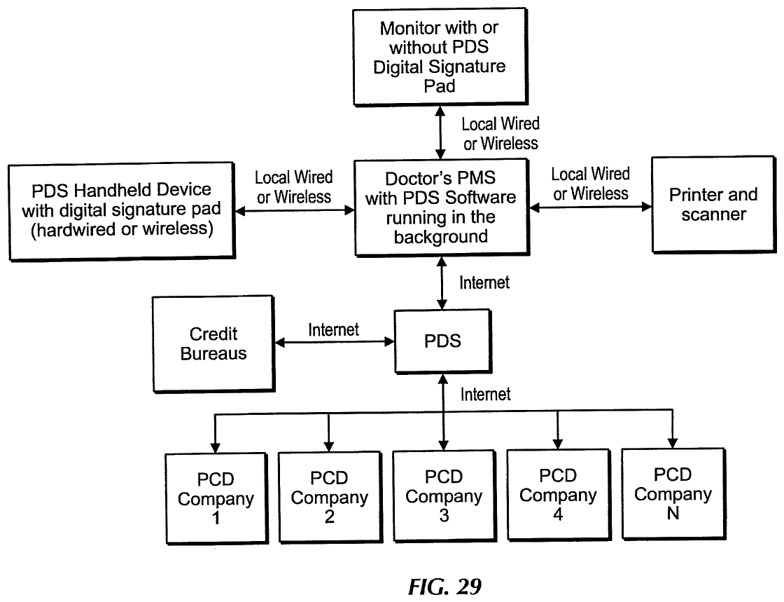

FIGS. 28 and 29 are block diagrams of the Patient Financing System (or sometimes referred to as the Direct Consumer Debt Revenue Cycle or DCDRC)) enabled by the PDS platform, under an embodiment. The platform is electronically coupled to one or more computers or systems at a treatment facility (e.g., doctor), and to third-party consumer debt providers via a consumer debt aggregator portal. In an alternative embodiment, a patient using a personal computer may be provided access to the platform to complete an application in advance of visiting the doctor. The coupling includes any type or combination of network technologies.

Embodiments of a Patient Financing System (PFS) are described herein. Generally, the PFS comprises one or more of platforms, systems, devices, applications or software, and an electronic site on the World Wide Web that collectively enables the electronic coupling or connection of healthcare providers, and/or patients to a spectrum of PCD financing companies. Example embodiments presented herein comprise the PFS integrated with the PDS either by being hosted on the PDS platform or coupled or connected through the PDS platform. Consequently, the PFS provides processes that efficiently and electronically facilitate the communication between patients and PCD financing companies, thereby providing the best opportunities for patients seeking financing of healthcare procedures.

The PFS provides patients with an optimal opportunity for financing of healthcare procedures given the credit worthiness of the patient. The systems and methods of the PFS include, in an embodiment, a network-based electronic infrastructure for coupling or connecting patients to a spectrum of PCD financing companies. Generally, the PFS of an embodiment electronically couples or connects treatment facilities or healthcare providers (e.g., medical doctors, providers, etc.) and their patients to multiple PCD financing companies. FIG. 29 is a block diagram of the Patient Financing System (PFS), under an embodiment. The PFS of an embodiment includes the PFS Platform and PFS interface where, under an embodiment, the PDS includes the PFS platform and the PDS interface includes and/or links to the PFS interface. The PFS Platform runs remotely (e.g., web-based, application service provider (ASP), etc.) and is coupled or connected to a healthcare provider (e.g., treatment facility, hospital, doctor's office, etc.) and the facility patient management system (PMS), credit bureaus, and PCD companies.

Alternatively, the PFS comprises PFS Software or Applications that run locally, for example, in the background of the healthcare provider practice management system (PMS), but the embodiment is not so limited; in this embodiment the PFS software is client-side software that communicates with the PDS platform. The PFS of another alternative embodiment includes a PFS Handheld Device (HHD) that can be coupled or connected via a wired and/or wireless coupling to the computer system at the healthcare provider. The PFS HHD comprises one or more of a keyboard, digital signature entry and capture device or component, LCD touch screen, PFS Software, and connectivity to the PFS Platform/Website via the network (e.g., Internet, etc.). The PFS also comprises a PFS Digital Signature Pad for accepting a patient's signature.

The PFS Software and graphic user interface (GUI) allows the healthcare provider to specify their approach to helping patients procure financing of medical or provider procedures using PCD financing. There are numerous credit application options and/or strategies that the healthcare provider and/or patient can select via the PFS including, but not limited to, the following: sending the PFS CAF to all PCD companies; selecting specific PCD companies to direct the patient's PFS CAF; sending the patient's PFS CAF based on the patient's FICO score, wherein the patient's FICO score meets or exceeds the minimum funding FICO score for PCD financing companies; and, unbundling treatment plans into smaller treatment plans for financing by more than one PCD financing company.

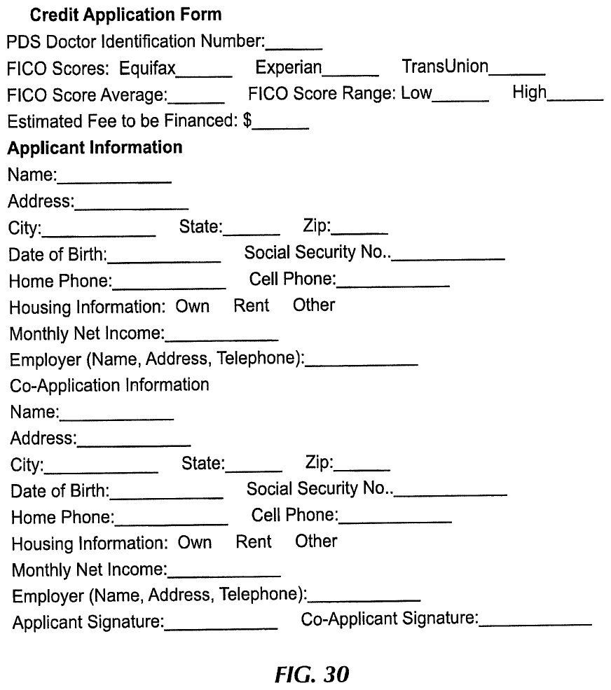

The PFS streamlines the process of seeking financing by healthcare providers and their patients. In particular, the PFS comprises a PFS CAF that electronically imports patient name, address, and other information from the healthcare provider PMS and automatically populates the PFS CAF using the imported PMS data or information. FIG. 30 is an example electronic CAF of the PFS, under an embodiment. The patient can review a digital copy of the PFS CAF on a computer monitor or PFS HHD. Alternatively, the PFS CAF can be printed, reviewed, and in some cases signed, then the paper CAF can be converted to an electronic format (e.g., digitized) and converted via the PFS into the PFS CAF digital format. Corrections or additions to the PFS CAF can be made and the patient can use the PFS digital signature pad locally connected to the healthcare provider's computer system and usually alongside the monitor or the PFS HHS digital signature capture capabilities.

The signed PFS CAF is uploaded to the PDS and is ready to be processed through the PDS to the aggregated PCD financing companies. According to the needs of the patient, the PFS GUI allows the healthcare provider's office to choose Selective or Non-Selective processing of the patient's PFS CAF to the aggregated PCD financing companies connected to the PFS Website, as described in detail below.

In general, Non-Selective processing of the PFS of an embodiment electronically pushes the patient PFS CAF (Jane Smith for example) downstream via the network or internet to the credit bureaus to capture the patient FICO score, then to all of the PCD financing companies for review of the patient's credit worthiness using the PFS CAF and the captured FICO score. In some cases, PCD financing companies require the CAF in the PCD specific format. The PFS platform comprises templates to convert the PFS CAF to a CAF specific to the PCD financing company.

Non-Selective processing of the PFS of an alternative embodiment electronically pushes the patient PFS CAF (Jane Smith for example) downstream via the network to the PCD financing company, which in turn, procures the patient FICO score. The PFS platform comprises templates to convert the PFS CAF to a CAF specific to one or more recipient PCD financing companies.

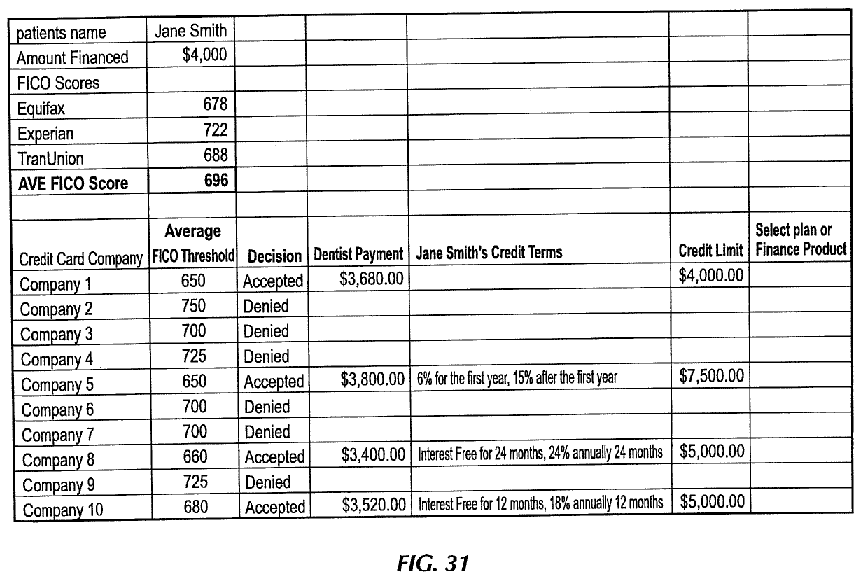

The PCD financing companies return a decision of whether or not to extend credit after reviewing the patient CAF received via the PFS. These decisions by the PCD financing companies are shown or presented by the PFS Website when logged into the PFS Website, wherein the financing decisions are displayed on a computer monitor or PFS HHD. FIG. 31 is an example PFS presentation of the results of the PFS process for an example involving a provider that is a dentist, under an embodiment. In the cases where credit is extended, the terms of the underlying credit products are displayed for the patient and/or healthcare provider to select.

When a patient selects a credit provider (or PCD financing company), the patient signs a PFS credit acceptance form using the PFS digital signature pad, and the signed PFS credit acceptance form is electronically sent to the PCD financing company. Similar to the PFS CAF and mapping of the PFS CAF to specific PCD financing company's CAF, the PFS comprises templates to map the PFS credit acceptance form to PCD financing companies' credit acceptance form. The PCD financing companies that approved credit for the patient but were not selected by the patient can be notified of the patient's decision electronically via the PFS. Alternatively, the healthcare provider can first view the finance products as result of the PCD financing companies accepting the patient's credit application and select the appropriate finance product before the patient selects the final finance product.

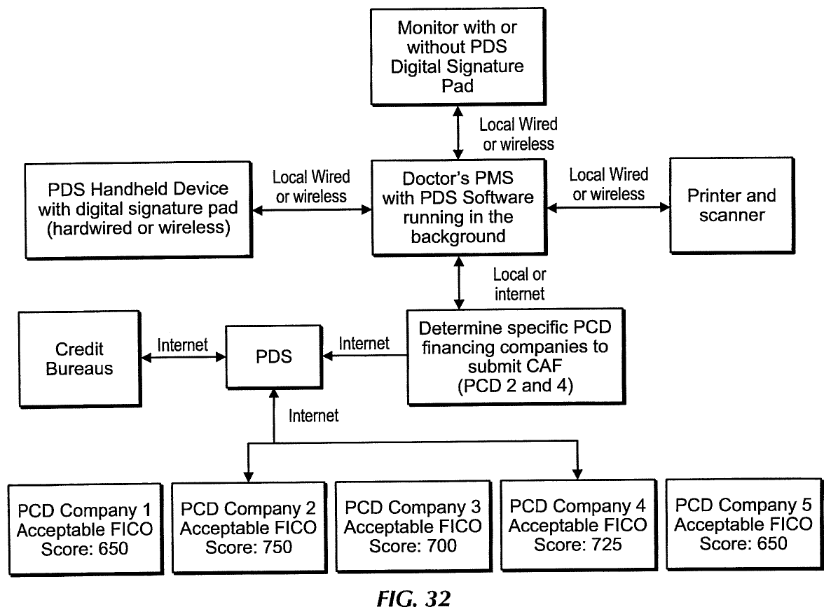

In general, the Selective processing of the PFS of an embodiment allows the patient or healthcare provider in the treatment example herein to electronically direct the patient's PFS CAF to specific PCD financing companies. FIG. 32 is a flow diagram for Selective processing of the PFS, under an embodiment. For example, with the patient's consent, the healthcare provider desires to submit the patient PFS CAF to two particular PCD financing companies, and the healthcare provider selects the appropriate PCD financing companies with which to begin processing.

In an embodiment of the PFS, the patient's FICO score is procured once from one or more credit bureaus, electronically attached or integrated into the patient's PFS CAF, and selectively or non-selectively transferred electronically to PCD financing companies aggregated on the PFS Website. Using the PFS, a patient's credit application along with a set of FICO scores and an amount to be financed is provided to one or more PCD companies based on a number of parameters including but limited to FICO score, amount to be financed, and combination of FICO scores. The PFS therefore enables unbundling of healthcare treatment plans into incrementally smaller and less expensive treatment plans, such that credit applications can be transmitted to one or more PCD companies for financing of each unbundled healthcare treatment plan.

In an alternative embodiment, the FICO score is procured by having the patient complete the PCD CAF via the PFS platform. The PFS software imports the patient's information (e.g., name, address, occupation, social security number, etc) from the healthcare provider's practice management system. The patient reviews the PFS CAF for correctness and completes or provides any missing information. The patient selects the PCD financing company or companies through the PFS platform. The PFS software includes a template to convert the PFS CAF to the PCD company-specific CAF. The patient signs or approves the PCD financing companies using the PFS digital signature pad or prints the CAF and signs the paper CAF, which could then sent via facsimile or email in PDF format. Under this alternative embodiment, the PCD financing sends or transfers the patient's information to a national credit bureau(s).

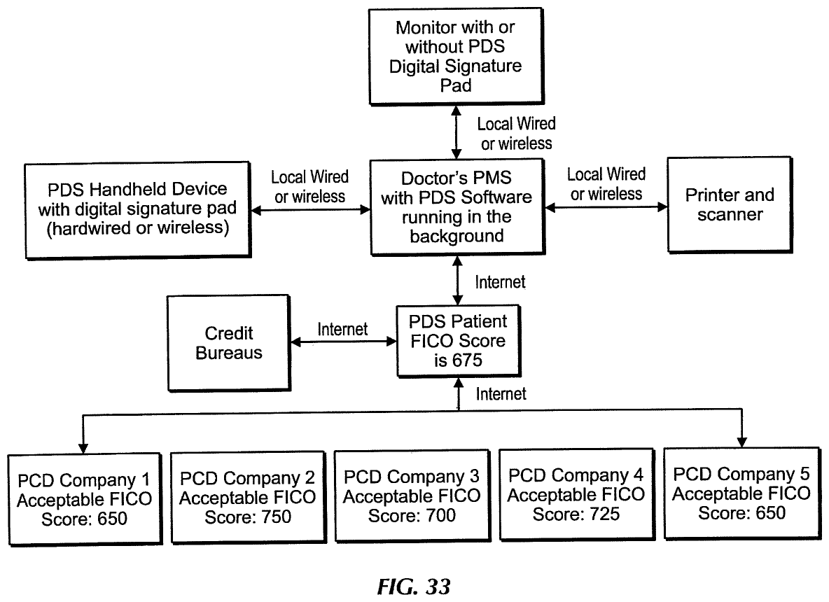

The PFS via the PFS Website can send one PFS CAF with FICO score(s) to one PCD financing company or simultaneously to multiple PCD financing companies. In another scenario, the PFS CAF with FICO score(s) can be selectively sent via the PFS Platform to PCD financing companies wherein the minimum FICO score threshold required to extend credit is known (FICO Score Filtering in FIG. 33). For example, and with reference to FIG. 31, if a PCD financing company is likely to finance a healthcare treatment plan amount up to $10,000 for FICO scores above 675 and a patient has a $4,000 provider procedure to finance with a FICO score of 690, then the PFS would send the PFS CAF to the PCD financing company with FICO funding thresholds of 690 or less (e.g., PCS Companies 1 and 5). If, on the other hand, the patient's FICO score is 640, then PFS will not send the credit application to any of the PCS companies presented and would return a message stating the patient's FICO does not meet the minimum funding level for the PCD financing companies selected.

The PFS devices, methods, and processes are exemplified and outlined using the provider example outlined above with reference to FIGS. 29, 30, 31, and 32. The PFS Software can reside on one or more of the PDS platform and the healthcare provider computer system (e.g., PMS). The healthcare provider can also log into the PFS via the Internet and run the PFS Software remotely from the Website. When a patient desires to seek third party financing for the $4,000 out-of-pocket costs, then the healthcare provider office administrator clicks on the PFS icon and starts the PFS financing process. Alternatively, the healthcare provider office administrator can be working in the PMS and click the PFS icon and begin working simultaneously and, for example, export the patient name, address, and social security number (if available) from the PMS to the PFS CAF. The healthcare provider information (number) is automatically populated on the PFS CAF. FIG. 30, described above, includes a sample of the PFS CAF.

The PFS Software comprises CAF and credit acceptance form templates corresponding to the PCD financing companies. If the PCD financing company prefers to have the CAF submitted, for example, in a specific format, then the PFS Software maps from the PFS CAF to the PCD financing company CAF.

The patient's information can be exported from the PMS to the PFS CAF where it is used to populate the CAF. Any information missing on the PFS CAF following the auto-population event can be entered via the computer system or the PFS HHD. The patient reviews the PFS CAF, corrects, updates, or adds any additional information, and signs the PFS CAF using the digital signature pad. After signature, the PFS will prompt the healthcare provider office whether for a number of items prior to sending the PFS CAF to all of the PCD financing companies (Non-Selective Process) or to set filter parameters for Selective Processing.

The PFS CAF with the digital signature is sent via the Internet to the PDS with the electronic instructions provided by the healthcare provider office and patient (Non-Selective Processing in this example). The patient name, address, and social security number is sent to the credit bureaus by the PDS, and a FICO credit score is returned to the PDS and attached to the PFS CAF. Prior to sending the PFS CAF with FICO score to the PCD financing companies, the PFS maps the treatment facility's PFS identification number to the specific PCD financing company's healthcare provider identification number. Each PFD CAF with the specific healthcare provider identification number is sent to the corresponding PCD financing company. Each PCD financing company reviews the patient PFS CAF and returns their decision to the PDS, which then securely displays the results at the provider office. As described above, FIG. 31 shows the result of processing a patient's (Jane Smith) $4,000 out-of-pocket costs for provider (dentist) treatment.

The PFS operations or processes of an embodiment comprise coupling or connecting from the provider office computer system to the PDS Software and/or PDS platform. At the level of the healthcare provider office, the administrator enters the PDS and prompts the administrator to select the type of credit application submission (Selective or Non-Selective) via the PFS and populate the PFS CAF. From either the PFS or the PMS, the PFS CAF is populated with the patient's name, address, and other information. The PFS CAF is reviewed by the provider office and patient, and any additional information is added and amended. Review of the PFS CAF is accomplished by printing the PFS CAF after the patient's data has populated the application and allowing the patient to complete or review the PFS CAF. Upon successful completion of the CAF, the patient uses the PFS Electronic Signature Pad and signs the PFS CAF.

Alternatively, review of the PFS CAF is accomplished using the PFS HHD. The patient completes and reviews the PFS CAF via the PFS HHD. The patient signs the PFS CAF using the PFS HHD once the PFS CAF is completed.

The completed PFS CAF is electronically sent from the healthcare provider to the PDS. The PDS sends the patient name, address, and social security number to the credit bureaus and, in response, the FICO score is returned to the PDS and added to the PFS CAF. Before electronically sending the patient PFS CAF to PCD financing companies, the PDS completes the PFS CAF by matching and mapping the healthcare provider PFS identification number to the healthcare provider identification number with the PCD financing companies that are to review the patient CAF. In some cases, PCD financing companies prefer to review the CAF using their designated CAF. As such, the PDS can map the patient PFS CAF to a PSC financing company-specific CAF.

The PDS transmits the PFS CAF (including patient FICO score, amount to be financed, and healthcare provider identification number) to PCD financing companies. PCD companies review the patient PFS CAF and return their decisions. The PDS sends the PCD financing company's decisions to the healthcare provider where they can be viewed via the GUI. The decision from PCD companies would include terms of any financing offered.

The patient, either using the PFS HHD or a computer at the healthcare provider, can select the finance product offered by any of the PCD financing companies approving the patient's credit application. Once selected, the selected provider of consumer debt financing is notified via the PFS. The selected provider of consumer debt financing acknowledges the acceptance and reconciles the financed showing the gross amount financed less fees and discounts (net amount financed). The healthcare provider receives the net amount financed, and the patient receives a statement showing the gross amount financed.

There are numerous variations to the PFS process described herein. For example, the PFS of an embodiment comprises FICO Score Filtering. FIG. 33 is a flow diagram of FICO Score Filtering of the PFS, under an embodiment. Following is an example of FICO Score Filtering under an embodiment. The minimum acceptable FICO score by PCD financing companies can be stored in the PDS Platform (see FIGS. 31 and 32 described above for examples of the minimum FICO acceptance or threshold scores). Once the patient FICO score is received at the PDS Platform, the PFS CAF can be selectively sent only to PCD companies for which the patient's FICO score is in the acceptance range. For example, if the patient's FICO score is 675, then the PFS FICO filtering algorithm enables only PCD companies 1 and 5 as the candidate companies that match the patient FICO score with their minimum acceptable FICO score. The FICO Score Filtering can be automatic, or set at the level of the healthcare provider when the PFS CAF is processed and transmitted to PCD financing companies for credit evaluation. The PFS FICO Score Filtering centers on the processing cost at the level of the PCD companies such that patient's that would not quality based on a PCD financing company's threshold acceptance score are not processed by that particular PCD financing company.

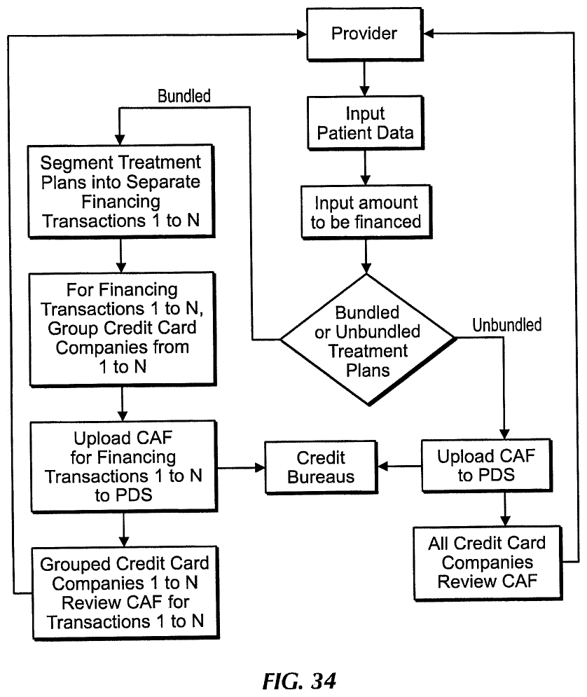

The PFS of an embodiment comprises Selective Standard Submission with Unbundling and FICO Score Filtering. FIG. 34 is a flow diagram of unbundling and selective processing of healthcare treatment plans, under an embodiment. Using the example presented above, there are treatment instances when out-of-pocket costs exceed $4,000. For example, partial or full mouth reconstruction using provider implants can cost the patient $25,000, or more with little or no provider insurance coverage. The PFS of an embodiment comprises several different processes for unbundling and processing the patient PFS CAF. For example, the $25,000 procedures can be divided into two provider treatment plans, one for $10,000 and the other for $15,000. The two unbundled treatment plans can be sequentially submitted after presenting the $25,000 provider treatment plan to the patient. After completing the PFS CAF for the $10,000 of the unbundled $25,000 treatment plan, the patient's PFS CAF is processed. The Selective process can be used under which specific PCD financing companies selected, but the embodiment is not so limited.

Alternatively, the FICO can be procured and the FICO Score Filter process can be selected and modified by selecting the specific PCD financing companies to which the $10,000 credit application is submitted (Modified FICO Score Filtering). In this example, the Modified the FICO Score Filtering process is invoked. Accordingly, the patient FICO is procured, matched with PCD financing companies wherein the FICO score meets or exceeds the PCD companies' threshold FICO score, and this result returned to the healthcare provider for evaluation and selection. The healthcare provider selects a set of PCD financing companies to submit the patient's $10,000 credit application. The patient's CAF is processed via the PFS and the results returned to the healthcare provider for evaluation by the healthcare provider and/or patient.

Once the credit application process for the unbundled $10,000 provider treatment plan is completed, the unbundled $15,000 provider treatment can be submitted. When submitting the $15,000 provider plan for financing, the patient and/or provider office would select a different set of PCD financing companies to review the credit application when compared to the unbundled $10,000 credit application. In summary, if a PCD financing company finances the $10,000 provider treatment plan, then the second unbundled $15,000 treatment plan of the $25,000 provider treatment plan is processed using the FICO Score Filter and a set of prospective PCD financing companies that are manually selected to be different from the PCD financing companies evaluating the unbundled $10,000 provider treatment plan. The $15,000 provider treatment plan is evaluated by the proposed PCD financing companies and the decision returned.

In some cases, PCD financing companies may openly and/or competitively bid on the financing of healthcare procedures for certain patients. The patient PFS CAF with the FICO score is posted securely on the PFS interface to be viewed by PCD financing companies. The PFS interface via connectivity to PCD financing companies can facilitate a bidding process for terms to provide financing, and the patient subsequently selects financing terms.

In some cases, PCD financing companies may elect not to participate (opt out) in the PFS process to finance healthcare procedures. The PFS can connect to the PCD financing company opting out and send the PFS CAF with or without the patient FICO score.

An element of the PFS (or DCDRC) includes, but is not limited to, completing the DCDRC patient application in the provider office. The CAF of the PFS (FIG. 30) can be entered digitally by the patient at a computer terminal or completed using a paper application. At times, the provider office has the patient information in digital format (for example, in the case of a practice management system integration) and can be imported. Using this method, the patient reviews the CAF and signs the electronic application using the digital signature pad or digital signature feature in the PFS. If the CAF is paper, provider office personnel manually enter the information into the computer. The patient reviews the CAF for correctness and uses a digital signature pad to sign the DCDRC application.

Completed CAF's are electronically sent to the platform via the web portal accessible by the provider office. The CAF is submitted to the portal aggregating providers of consumer debt. The platform submits certain aspects of the CAF the credit agencies to procure the patient's FICO score. After the patient's credit worthiness is scored, the CAF is submitted through the platform to the portal aggregating providers of consumer debt.

Providers of consumer debt review the patient's CAF and respond to the provider office via the platform. The patient selects a financing option from a menu of financing products approved by the provider(s) of consumer debt (FIG. 31). The selected finance product is returned to the selected provider of debt via the platform.

Funding of the provider treatment plan or procedure is provided and electronically transferred to the provider account directly or in the platform. The corresponding credit card and statement is sent directly to the patient from the provider of consumer debt financing. The platform charges a fee for processing the CAF through the PDS platform and web portal.

Provider offices can integrate the PDS platform into the provider practice via a subscription or per use fee. The PDS can run or be accessed in the background of provider practice management systems or other products, via a web portal, and/or as a stand-alone offering.

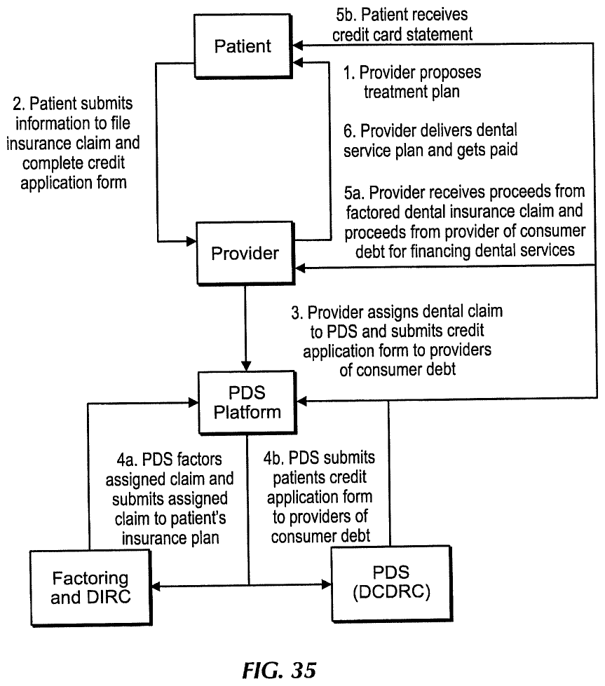

FIG. 35 is a block diagram of the provider using the platform to facilitate the delivery of provider services by factoring the assign provider claim and seeking patient financing for the proposed provider treatment plan through the PFS enabled by the platform, under an embodiment. The combined DIRC and PFS simultaneously process provider insurance and patient financing. The platform is electronically coupled to one or more computers or systems at a treatment facility (e.g., doctor) and a DIRC platform and a DCDRC platform. In an alternative embodiment, a patient using a personal computer may be provided access to the platform. The coupling includes any type or combination of network technologies.

The platform of an embodiment assists providers and their provider practices with practice cash flows and in particular the cash sources of their working capital management. From the perspective of the revenue contribution of the provider income statement and working capital management, sources of cash to the provider practice are derived from cash or check paid by the patient, patient credit card, provider insurance, patient financing via providers of unsecured consumer debt financing, and/or credit extended by the provider via patient billing, to name a few. The platform converts accounts receivable to cash and compresses accounts receivable to the level of credit extended by the provider to patients from patient billing by the provider office.

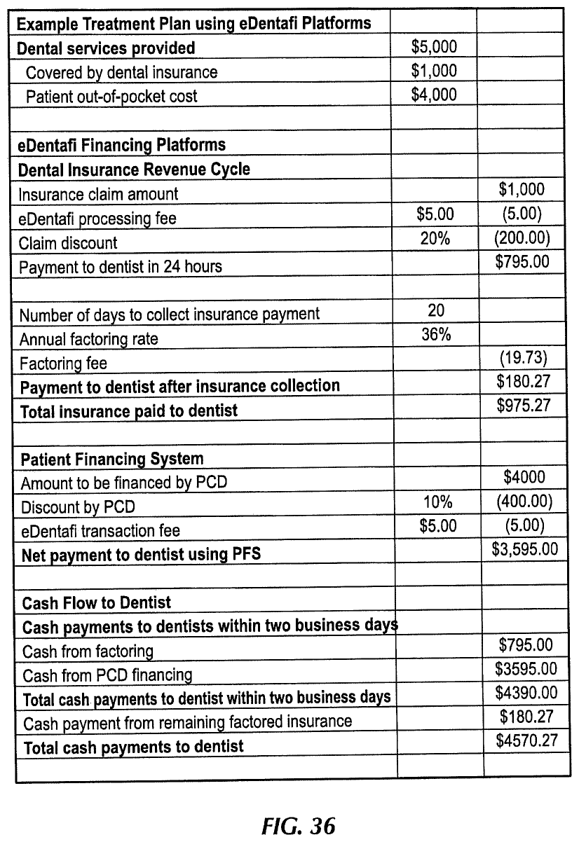

FIG. 36 shows an example of a patient seeking the services of a provider (dentist) that is recommending a $5,000 provider treatment plan, $1,000 of which is covered by the patient's provider insurance plan and the remaining $4,000 would be required to be covered by the patient as an out-of-pocket cost, under an embodiment. The patient elects to move forward with the proposed provider treatment plan if they could secure financing through the PFS of the platform. The patient completes the CAF, which is submitted to providers of consumer debt via the PFS of the platform. The patient's CAF is approved by a PCD and the remaining $4,000 of the proposed treatment plan is financed. This entire process to assign the provider claim, submit the provider claim to the provider insurance plan, complete and submit the patient's CAF through the PFS is very efficient and takes on the order of several minutes in the provider office. The benefits of the platform streamlines the delivery of provider services, provides a service to the patient by facilitating their provider treatment, allows the provider to deliver the treatment plan to treat the patient's provider condition, streamlines back office financial processes, and increases the provider's service revenues. More specifically, the provider, using the platform, delivers $5,000 in provider services to the patient that he/she would not have otherwise delivered. In addition, the provider would receive $4,390.00 of the $5,000 provider service within about 2 business days with the remaining $187.20 in about 20 after factoring the assigned provider claim.

The platform of an embodiment includes the PDS and the PFS as described in detail herein, and one example of the platform is the system provided by CirraGroup LLC of Lafayette, La. (CirraGroup). An example of the PDS is CirraCheck, and an example of the PFS is CirraFi, both provided by CirraGroup. The patient data of an embodiment is collected by CirraCheck, which is a credit reporting agency. As a credit reporting agency, CirraCheck is limited by HIPAA, 45 CFR 164.501, to the type of data that can be collected (e.g., no diagnosis or procedure codes and limited demographic data). This can limit the data's usefulness later in the collection cycle because the patient can be shown that they have outstanding balances with providers, but they cannot be shown information regarding services performed that correspond to the charges. This can make it more difficult to collect data from providers since conventional data export formats include data that is not allowed to be collected by a credit-reporting agency. Additionally, many providers incur costs in generating non-standard data exports.

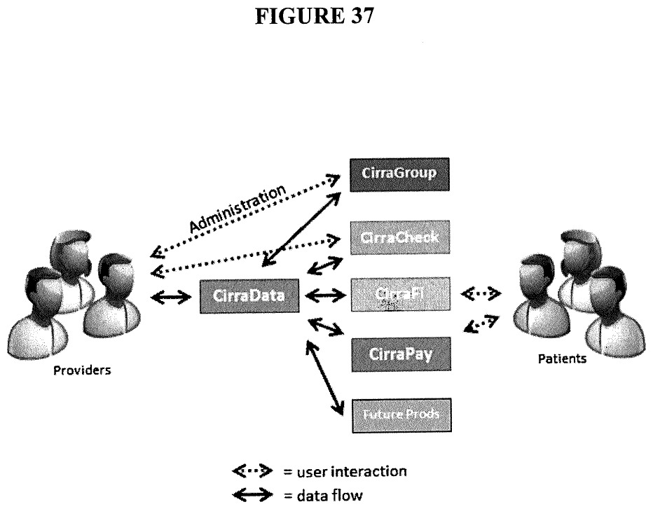

FIG. 37 is a block diagram of patient revenue cycle platform, under an alternative embodiment. The platform of this embodiment includes an application or component as the database that is not characterized as or under control of a credit reporting agency. This application or component, referred to herein as CirraData, is a component of CirraGroup, which is not a credit reporting agency. CirraData therefore is an intermediary configured to receive patient data without any restrictions relating to credit reporting agencies, including PII and PHI is received from the healthcare providers. CirraData is further configured to filter or control access to the data as appropriate to requesting components or entities, and the filtered data is then provided to the requesting component.

This platform configuration means that CirraGroup, because it is not a credit reporting agency, is not encumbered by the limitations on the type of data that it can collect. Therefore, richer patent data (e.g., PHI, diagnosis codes, procedure codes, etc.) can be received and maintained by CirraData, thereby removing current restrictions on healthcare providers as to the data they export. Furthermore, this allows patients to be provided richer data on debts and outstanding amounts owed to healthcare providers so that they are better informed regarding monies owed by patient to providers. Consequently, use of a single database for maintaining all patient data simplifies the provider data transfer to a single data transfer to CirraData. The platform subsequently administers any necessary data filtering as well as controlling access to the data so that a component is only allowed to access data configured and filtered for that component.

The platform of an embodiment includes an application or component, referred to herein as CirraPay, which is a patient payment portal for subscribing physicians. As a payment portal, CirraPay is configured to provide a gateway for data into the CirraCheck/CirraFi system once the charges become delinquent. The platform of an embodiment is configured to enable a patient to access the CirraFi and CirraPay components of the platform.

Embodiments include separate databases dedicated to one or more of the platform components (e.g., CirraCheck, CirraFi, CirraPay, etc.). In this manner, data necessary only to the corresponding component (e.g., configuration data, etc.) is stored in the database corresponding to that component.

CirraData comprises or is coupled to application programming interfaces (APIs) that are configured to limit access to the type of data accessible by other applications or companies. For example, CirraCheck will be unable to access diagnosis or procedure codes from CirraData when searching for debt data, while other components (e.g., CirraPay, CirraFi, etc.) described herein can access the related codes from CirraData but only for the patient requesting the search.

Each component of the platform has access to an API corresponding to that component, and the API controls or limits the data that the corresponding component can access. Therefore, the platform includes a first API that corresponds to a first component (e.g., CirraCheck), and the first API controls or limits access by the first component to a first set of data that comprises only the data allowed to be accessed by the first component. Likewise, the platform includes a second API that corresponds to a second component (e.g., CirraFi), and the second API controls or limits access by the second component to a second set of data that comprises only the data allowed to be accessed by the second component, with the second data set including different types of data than the first data set. Moreover, the platform includes a third API that corresponds to a third component (e.g., CirraPay), and the third API controls or limits access by the third component to a third set of data that comprises only the data appropriate to be accessed by the third component.

The APIs are also accessible by third party applications or systems outside the platform. For example, a third party involved with collection of overdue balances accesses the appropriate data of the platform via an API that controls access by the third party to only the data allowed to be accessed by the third party. As another example, a third party involved with providing financing to patients for medical debt accesses the appropriate data of the platform via an API that controls access by that third party to only the data allowed to be accessed by that third party.

The platform of an embodiment includes an application or component, referred to herein as CirraExchange, which is installed on and runs on a computer at the healthcare provider offices and securely exchanges data between the healthcare provider facilities and the CirraGroup cloud. CirraExchange is configured to enable a healthcare provider to indicate or select data on their system that is to be transferred to the platform, and to automatically and securely transfer the selected data to the platform over a network coupling with the platform. The transferred data is received at CirraData as described in detail herein.

The platform of an embodiment is configured to electronically communicate with a practice management system (PMS) of a healthcare provider. A third party involved practice management system (PMS) accesses the appropriate data of the platform via an API that controls access by the PMS to only the data allowed to be accessed by the PMS, but is not so limited. Additionally, the PMS user interface of an embodiment includes an icon or link to the PMS API of the platform.

The platform provides the components or applications described herein as components of a portfolio that seamlessly adds functionality to providers in different areas of the revenue cycle. When healthcare provides subscribe to the platform services, the platform of an embodiment is configured to allow the providers to select the components to which they subscribe. The platform therefore comprises a central electronic site (“CirraGroup”) including a user interface that includes access to all administrative and configuration controls of all components so that a provider can access this central site to configure all components of the platform to which they subscribe instead of having to separately access and configure each component. A healthcare provider can also supplement their subscription to additional components via this central site.

Embodiments include a system comprising a platform including a processor. A database application running on the platform is configured to receive patient data from a plurality of providers and generate a database including the patient data. The patient data includes identification data and healthcare data of a plurality of patients. A first application running on the platform is configured to receive from the plurality of providers first electronic queries of the database and, in response, generate from the patient data a first set of data comprising data of delinquent transactions. A second application running on the platform is configured to receive from the plurality of patients second electronic queries of the database and, in response, provide a second set of data corresponding to the second electronic query and present a payment interface configured to receive payments at the platform according to a plurality of payment options. The payments correspond to transactions of the plurality of providers. A third application running on the platform is configured to receive from the plurality of patients third electronic queries of the database and, in response, provide a third set of data corresponding to the third electronic query, a plurality of finance options, and present a finance interface configured to associate requesting patients with at least one source of debt financing for payment of the delinquent transactions.

Embodiments include a system comprising: a platform including a processor; a database application running on the platform and configured to receive patient data from a plurality of providers and generate a database including the patient data, wherein the patient data includes identification data and healthcare data of a plurality of patients; a first application running on the platform and configured to receive from the plurality of providers first electronic queries of the database and, in response, generate from the patient data a first set of data comprising data of delinquent transactions; a second application running on the platform and configured to receive from the plurality of patients second electronic queries of the database and, in response, provide a second set of data corresponding to the second electronic query and present a payment interface configured to receive payments at the platform according to a plurality of payment options, wherein the payments correspond to transactions of the plurality of providers; and a third application running on the platform and configured to receive from the plurality of patients third electronic queries of the database and, in response, provide a third set of data corresponding to the third electronic query, a plurality of finance options, and present a finance interface configured to associate requesting patients with at least one source of debt financing for payment of the delinquent transactions.

The identification data includes Personally Identifiable Information (PII).

The healthcare data includes Protected Health Information (PHI).

The system includes a provider application coupled to the platform, wherein the provider application is hosted by each of the plurality of providers, wherein the provider application is configured to control selection and transmission of patient data of a provider to the database application.

The provider application includes a practice management system of the provider.

The platform is configured to limit access to the patient data by the first application, the second application, and the third application.

The patient data includes account data of patients having delinquent accounts with the plurality of providers.

The platform is configured to credit an amount of a payment to an account of a provider corresponding to a delinquent transaction of a patient.

The platform is configured to update the database to include data of the credit.

The database application is configured to receive updated patient data from the plurality of providers and, in response, update the database.

The updates comprise removing an unpaid amount from the database in response to receiving payment of the unpaid amount.

The patient data includes patient identification data.

The patient data comprises at least one of name, address, date of birth, and social security number of patient debtors.

The patient data includes financial data of a corresponding healthcare transaction.

The patient data includes insurance data.

The patient data includes names of provider creditors.

The patient data includes location of the providers.

The first set of data includes a service date and an unpaid amount corresponding to the delinquent transactions.

The system includes a fourth application configured to receive assignment of a plurality of claims corresponding to the plurality of patients.

The platform is configured to submit the plurality of claims to a plurality of payers.

The platform is configured to generate a plurality of discounts of the plurality of claims.

The platform is configured to generate a payment to a corresponding provider in an amount of the corresponding claim less the corresponding discount.

The platform is configured to generate each discount using factors that include at least one of a score of the corresponding provider, an amount of the claim, and an insurance policy corresponding to the patient, wherein the score comprises a reimbursement metric of the provider.

The platform is configured to determine the score using one or more attributes of the provider.

The one or more attributes comprise type of the provider, location of the provider, information of a patient population of the provider, information of insurance coverage of at least one member of the patient population, insurance reimbursement processes of the provider, information of at least one of current and historical aged insurance receivables, number of claims submitted by the provider for reimbursement, and reimbursement levels relating to the submitted claims.

The system includes tracking adjudication of the claim by the payer and providing status data of the adjudication.

The system includes reconciling the payment to the provider and funds received from the payer and generating the updated transaction data to include final data of the adjudication.

When the claim is denied the reconciling comprises reconciling an amount of the payment with funds received from the payer for at least one subsequent claim of the corresponding provider.

When the claim is partially paid the reconciling comprises reconciling a difference between the payment and an amount of the partial payment from the payer with at least one subsequent claim of the provider.

The system includes at least one remote application, wherein the at least one remote application is hosted on a third party system, wherein the platform is configured to limit access to the patient data by the at least one remote application.

Embodiments include a method comprising receiving via a database application running on a platform patient data from a plurality of providers and generating a database including the patient data. The patient data includes identification data and healthcare data of a plurality of patients. The method includes receiving from a plurality of providers via a first application running on the platform first electronic queries of the database and, in response, generating from the patient data a first set of data comprising data of delinquent transactions. The method includes receiving from a plurality of patients via a second application running on the platform second electronic queries of the database and, in response, providing a second set of data corresponding to the second electronic query and presenting a payment interface configured to receive payments at the platform according to a plurality of payment options. The payments correspond to transactions of the plurality of providers. The method includes receiving from the plurality of patients via a third application running on the platform third electronic queries of the database and, in response, providing a third set of data corresponding to the third electronic query, a plurality of finance options, and presenting a finance interface configured to associate requesting patients with at least one source of debt financing for payment of the delinquent transactions.

Embodiments include a method comprising: receiving via a database application running on a platform patient data from a plurality of providers and generating a database including the patient data, wherein the patient data includes identification data and healthcare data of a plurality of patients; receiving from a plurality of providers via a first application running on the platform first electronic queries of the database and, in response, generating from the patient data a first set of data comprising data of delinquent transactions; receiving from a plurality of patients via a second application running on the platform second electronic queries of the database and, in response, providing a second set of data corresponding to the second electronic query and presenting a payment interface configured to receive payments at the platform according to a plurality of payment options, wherein the payments correspond to transactions of the plurality of providers; and receiving from the plurality of patients via a third application running on the platform third electronic queries of the database and, in response, providing a third set of data corresponding to the third electronic query, a plurality of finance options, and presenting a finance interface configured to associate requesting patients with at least one source of debt financing for payment of the delinquent transactions.

In the description above, numerous specific details are introduced to provide a thorough understanding of, and enabling description for, embodiments of the systems and methods. One skilled in the relevant art, however, will recognize that these embodiments can be practiced without one or more of the specific details, or with other components, systems, etc. In other instances, well-known structures or operations are not shown, or are not described in detail, to avoid obscuring aspects of the disclosed embodiments.

The systems and methods described herein include and/or run under and/or in association with a processing system. The processing system includes any collection of processor-based devices or computing devices operating together, or components of processing systems or devices, as is known in the art. For example, the processing system can include one or more of a portable computer, portable communication device operating in a communication network, and/or a network server. The portable computer can be any of a number and/or combination of devices selected from among personal computers, cellular telephones, personal digital assistants, portable computing devices, and portable communication devices, but is not so limited. The processing system can include components within a larger computer system.

The processing system of an embodiment includes at least one processor and at least one memory device or subsystem. The processing system can also include or be coupled to at least one database. The term “processor” as generally used herein refers to any logic processing unit, such as one or more central processing units (CPUs), digital signal processors (DSPs), application-specific integrated circuits (ASIC), etc. The processor and memory can be monolithically integrated onto a single chip, distributed among a number of chips or components of a host system, and/or provided by some combination of algorithms. The methods described herein can be implemented in one or more of software algorithm(s), programs, firmware, hardware, components, circuitry, in any combination.

System components embodying the systems and methods described herein can be located together or in separate locations. Consequently, system components embodying the systems and methods described herein can be components of a single system, multiple systems, and/or geographically separate systems. These components can also be subcomponents or subsystems of a single system, multiple systems, and/or geographically separate systems. These components can be coupled to one or more other components of a host system or a system coupled to the host system.

Communication paths couple the system components and include any medium for communicating or transferring files among the components. The communication paths include wireless connections, wired connections, and hybrid wireless/wired connections. The communication paths also include couplings or connections to networks including local area networks (LANs), metropolitan area networks (MANs), wide area networks (WANs), proprietary networks, interoffice or backend networks, and the Internet. Furthermore, the communication paths include removable fixed mediums like floppy disks, hard disk drives, and CD-ROM disks, as well as flash RAM, Universal Serial Bus (USB) connections, RS-232 connections, telephone lines, buses, and electronic mail messages.

Unless the context clearly requires otherwise, throughout the description, the words “comprise,” “comprising,” and the like are to be construed in an inclusive sense as opposed to an exclusive or exhaustive sense; that is to say, in a sense of “including, but not limited to.” Words using the singular or plural number also include the plural or singular number respectively. Additionally, the words “herein,” “hereunder,” “above,” “below,” and words of similar import refer to this application as a whole and not to any particular portions of this application. When the word “or” is used in reference to a list of two or more items, that word covers all of the following interpretations of the word: any of the items in the list, all of the items in the list and any combination of the items in the list.

The above description of embodiments of the systems and methods is not intended to be exhaustive or to limit the systems and methods described to the precise form disclosed. While specific embodiments of, and examples for, the systems and methods are described herein for illustrative purposes, various equivalent modifications are possible within the scope of other systems and methods, as those skilled in the relevant art will recognize. The teachings of the systems and methods provided herein can be applied to other processing systems and methods, not only for the systems and methods described above.

The elements and acts of the various embodiments described above can be combined to provide further embodiments. These and other changes can be made to the systems and methods in light of the above detailed description.

What is claimed is:

1. A system comprising:
a platform including a processor;
a database application running on the platform and configured to receive patient data from a plurality of providers and generate a database including the patient data, wherein the patient data includes identification data and healthcare data of a plurality of patients;
a first application running on the platform and configured to receive from the plurality of providers first electronic queries of the database and, in response, generate from the patient data a first set of data comprising data of delinquent transactions;
a second application running on the platform and configured to receive from the plurality of patients second electronic queries of the database and, in response, provide a second set of data corresponding to the second electronic query and present a payment interface configured to receive payments at the platform according to a plurality of payment options, wherein the payments correspond to transactions of the plurality of providers; and
a third application running on the platform and configured to receive from the plurality of patients third electronic queries of the database and, in response, provide a third set of data corresponding to the third electronic query, a plurality of finance options, and present a finance interface configured to associate requesting patients with at least one source of debt financing for payment of the delinquent transactions.

2. The system of claim 1, wherein the identification data includes Personally Identifiable Information (PII).

3. The system of claim 1, wherein the healthcare data includes Protected Health Information (PHI).

4. The system of claim 1, comprising a provider application coupled to the platform, wherein the provider application is hosted by each of the plurality of providers, wherein the provider application is configured to control selection and transmission of patient data of a provider to the database application.

5. The system of claim 4, wherein the provider application includes a practice management system of the provider.

6. The system of claim 1, wherein the platform is configured to limit access to the patient data by the first application, the second application, and the third application.

7. The system of claim 1, wherein the patient data includes account data of patients having delinquent accounts with the plurality of providers.

8. The system of claim 7, wherein the platform is configured to credit an amount of a payment to an account of a provider corresponding to a delinquent transaction of a patient.

9. The system of claim 8, wherein the platform is configured to update the database to include data of the credit.

10. The system of claim 1, wherein the database application is configured to receive updated patient data from the plurality of providers and, in response, update the database.

11. The system of claim 10, wherein the updates comprise removing an unpaid amount from the database in response to receiving payment of the unpaid amount.

12. The system of claim 1, wherein the patient data includes patient identification data.

13. The system of claim 1, wherein the patient data comprises at least one of name, address, date of birth, and social security number of patient debtors.

14. The system of claim 1, wherein the patient data includes financial data of a corresponding healthcare transaction.

15. The system of claim 1, wherein the patient data includes insurance data.

16. The system of claim 1, wherein the patient data includes names of provider creditors.

17. The system of claim 1, wherein the patient data includes location of the providers.

18. The system of claim 1, wherein the first set of data includes a service date and an unpaid amount corresponding to the delinquent transactions.

19. The system of claim 1, comprising a fourth application configured to receive assignment of a plurality of claims corresponding to the plurality of patients.

20. The system of claim 19, wherein the platform is configured to submit the plurality of claims to a plurality of payers.

21. The system of claim 19, wherein the platform is configured to generate a plurality of discounts of the plurality of claims.

22. The system of claim 21, wherein the platform is configured to generate a payment to a corresponding provider in an amount of the corresponding claim less the corresponding discount.

23. The system of claim 19, wherein the platform is configured to generate each discount using factors that include at least one of a score of the corresponding provider, an amount of the claim, and an insurance policy corresponding to the patient, wherein the score comprises a reimbursement metric of the provider.

24. The system of claim 23, wherein the platform is configured to determine the score using one or more attributes of the provider.

25. The system of claim 24, wherein the one or more attributes comprise type of the provider, location of the provider, information of a patient population of the provider, information of insurance coverage of at least one member of the patient population, insurance reimbursement processes of the provider, information of at least one of current and historical aged insurance receivables, number of claims submitted by the provider for reimbursement, and reimbursement levels relating to the submitted claims.

26. The system of claim 22, comprising tracking adjudication of the claim by the payer and providing status data of the adjudication.

27. The system of claim 26, comprising reconciling the payment to the provider and funds received from the payer and generating the updated transaction data to include final data of the adjudication.

28. The system of claim 27, wherein when the claim is denied the reconciling comprises reconciling an amount of the payment with funds received from the payer for at least one subsequent claim of the corresponding provider.

29. The system of claim 27, wherein when the claim is partially paid the reconciling comprises reconciling a difference between the payment and an amount of the partial payment from the payer with at least one subsequent claim of the provider.

30. The system of claim 1, comprising at least one remote application, wherein the at least one remote application is hosted on a third party system, wherein the platform is configured to limit access to the patient data by the at least one remote application.

31. A method comprising:
receiving via a database application running on a platform patient data from a plurality of providers and generating a database including the patient data, wherein the patient data includes identification data and healthcare data of a plurality of patients;
receiving from a plurality of providers via a first application running on the platform first electronic queries of the database and, in response, generating from the patient data a first set of data comprising data of delinquent transactions;
receiving from a plurality of patients via a second application running on the platform second electronic queries of the database and, in response, providing a second set of data corresponding to the second electronic query and presenting a payment interface configured to receive payments at the platform according to a plurality of payment options, wherein the payments correspond to transactions of the plurality of providers; and
receiving from the plurality of patients via a third application running on the platform third electronic queries of the database and, in response, providing a third set of data corresponding to the third electronic query, a plurality of finance options, and presenting a finance interface configured to associate requesting patients with at least one source of debt financing for payment of the delinquent transactions.
